(19) United States
(12) Patent Application Publication
(10) Pub. No.: US 2024/0336689 A1
(43) Pub. Date: Oct. 10, 2024

(54) TREATMENT OF CANCERS WITH AN ANTIBODY THAT BINDS LGR5 AND EGFR

(71) Applicant: MERUS N.V., Utrecht (NL)

(72) Inventors: Leonardo Andres SIRULNIK, Utrecht (NL); Ernesto Isaac WASSERMAN, Utrecht (NL)

(21) Appl. No.: 18/257,528

(22) PCT Filed: Dec. 15, 2021
(86) PCT No.: PCT/NL2021/050763

§ 371 Date: Jun. 14, 2023

(30) Foreign Application Priority Data
Dec. 15, 2020 (NL) 2027118

(51) Int. Cl. C07K 16/28; A61K 9/00; A61K 9/08; A61P 35/00

(52) U.S. Cl. CPC C07K 16/2863; A61K 9/0014; A61K 9/08; A61P 35/00; C07K 16/28; C07K 2317/31

(57) ABSTRACT

The disclosure relates to means and methods in the treatment of cancer. The disclosure in particular relates to a method of treating a cancer in an individual with an antibody that binds LGR5 and EGFR. The invention further relates to the combination for use in such methods and to the combination for use in the manufacture of a medicament for the treatment of head and neck cancer.

### FIELD OF THE INVENTION

The disclosure relates to means and methods in the treatment of cancer. The disclosure in particular relates to a method of treating a cancer in an individual with an antibody that binds LGR5 and EGFR. The invention further relates to the combination for use in such methods and to the combination for use in the manufacture of a medicament for the treatment of head and neck cancer. Such antibodies are particularly useful in the treatment of head and neck cancer.

### BACKGROUND OF THE INVENTION

Traditionally, most cancer drug discovery has focused on agents that block essential cell functions and kill dividing cells via chemotherapy. However, chemotherapy rarely results in a complete cure. In most cases, the tumors in the patients stop growing or temporarily shrink (referred to as remission) only to start proliferating again, some times more rapidly (referred to as relapse), and become increasingly more difficult to treat. More recently the focus of cancer drug development has moved away from broadly cytotoxic chemotherapy to targeted cytostatic therapies with less toxicity. Treatment of advanced cancer with targeted therapy that specifically inhibits signaling pathway components has been validated clinically in leukemia. However, in a majority of carcinomas, targeted approaches are still proving ineffective.

Cancer is still a major cause of death in the world, in spite of the many advances that have been made in the treatment of the disease and the increased knowledge of the molecular events that lead to cancer. It has been reported that, in the United States, head and neck cancer, in particular in the oral cavity and pharynx, already accounts for 3 percent of malignancies, with approximately 53,000 Americans developing such cancer annually and 10,800 dying therefrom (Siegel et al., CA Cancer J Clin. 2020; 70(1):7. Epub 2020 Jan. 8.). Furthermore, head and neck squamous cell carcinoma (HNSCC) is reported to be the sixth leading cancer by incidence worldwide, with a five-year overall survival rate of patients with HNSCC of about 40-50% (in Head and Neck Cancer, Union for International Cancer Control, 2014 Review of Cancer Medicines on the WHO List of Essential Medicines).

A meta-analysis into locoregionally advanced head and neck squamous cell carcinoma (LA-HNSCC) reported that the addition of an anti-EGFR agent to radiotherapy or chemoradiotherapy did not improve clinical outcomes in patients with LA-HNSCC (Oncotarget. 2017; 8(60):102371-102380). Also, the addition of anti-EGFR agents was reported to increase the risk of skin toxicities and mucositis.

A need thus exists for improved or alternative cancer treatments, in particular for treating head and neck cancer.

### SUMMARY OF THE INVENTION

The disclosure provides the following preferred aspects and embodiments. However, the invention is not limited to thereto.

The present disclosure provides an antibody or functional part, derivative and/or analogue thereof that comprises a variable domain that binds an extracellular part of EGFR and a variable domain that binds an extracellular part of LGR5 for use in the treatment of cancer in a subject, wherein said use comprises providing the subject with a flat dose of 1500 mg of the antibody or functional part, derivative and/or analogue thereof. The cancer to be treated preferably is head and neck cancer.

The disclosure further provides methods of treating head and neck cancer comprising administering to a subject in need thereof an antibody or functional part, derivative and/or analogue thereof that comprises a variable domain that binds an extracellular part of EGFR and a variable domain that binds an extracellular part of LGR5.

Also provided is the use of an antibody or functional part, derivative and/or analogue thereof, that comprises a variable domain that binds an extracellular part of EGFR and a variable domain that binds an extracellular part of LGR5 in the manufacture of a medicament for the treatment of head and neck cancer, wherein said use comprises providing or administering the subject with a flat dose of 1500 mg of the antibody or functional part, derivative and/or analogue thereof.

The disclosure provides an antibody or functional part, derivative and/or analogue thereof that comprises a variable domain that binds an extracellular part of EGFR and a variable domain that binds an extracellular part of LGR5 for use in the treatment of head and neck cancer in a subject. The disclosure further provides methods of treating head and neck cancer in a subject comprising providing the subject in need thereof with the antibody or functional part, derivative and/or analogue thereof. Preferably said use comprises providing the subject with a flat dose of 1500 mg of the antibody or functional part, derivative and/or analogue thereof.

Preferably, the antibody or functional part, derivative and/or analogue thereof is provided intravenously.

Preferably, the cancer has a mutation in one or more EGFR signaling pathway genes, more preferably HRAS, MAP2K1 and/or PLCG2. More preferably, the mutation is present in a gene the expression product of which is active downstream of EGFR in the EGFR signaling pathway, most preferably in HRAS.

Preferably, the cancer has a mutation in one or more WNT signaling pathway genes, more preferably in APC, CREPPB, CUL1, EP300, SOX17 and/or TP53.

Preferably, the cancer has a mutation in a gene selected from AKT1, KRAS, MAP2K1, NRAS, HRAS, PIK3CA, PTEN and EGFR. More preferably, the cancer has a mutation in a gene coding for TP53, PIK3CA, CDKN2A, NOTCH1, HRAS and/or MAP2K1. Preferably, the cancer has a mutation in one or more genes depicted in table 1. Preferably, the cancer has one or more of the mutations depicted in table 1.

In particular, the cancer is head and neck cancer, more in particular a squamous cell carcinoma or an adenocarcinoma, most in particular Head and Neck Squamous Cell Carcinoma (HNSCC). In particular, the head and neck cancer may occur in the pharynx. This includes the nasopharynx, the oropharynx, the hypopharynx. In particular, the head and neck cancer may occur in the larynx. In particular, the head and neck cancer may occur in the paranasal sinuses and nasal cavity. In particular, the head and neck cancer may occur in the salivary glands. In a preferred disclosure, the cancer is HNSCC of the oropharynx.

The head and neck cancer in particular thus includes an adenocarcinoma, but more preferably is a squamous cell carcinomas of the head and neck, such as nasopharyngeal cancer, laryngeal cancer, hypopharyngeal cancer, nasal cavity cancer, paranasal sinus cancer, oral cancer, oropharyngeal cancer and salivary gland cancer.

Preferably, the cancer expresses EGFR and/or expresses LGR5. As used herein, a cancer expresses LGR5 if the cancer comprises cells that express LGR5. A cell which expresses LGR5 comprises detectable levels of RNA that codes for LGR5. As used herein, a cancer expresses EGFR if the cancer comprises cells that express EGFR. A cell which expresses EGFR comprises detectable levels of RNA that codes for EGFR. Expression can also be detected by incubating the cell with an antibody that binds to LGR5 or EGFR, and through detection by use of immunohistochemistry against either or both antigens.

Preferably, the cancer expresses LGR5 in sufficient levels for an antibody to bind the LGR5 protein, especially an antibody comprising a VH chain of the variable domain that binds LGR5 that comprises the amino acid sequence of the VH chain of MF5816 as depicted in FIG. 3, or alternative variable domains that bind LGR5 set out herein. Preferably, the cancer expresses EGFR in sufficient levels for an antibody to bind the EGFR protein, especially an antibody comprising a VH chain of the variable domain that binds EGFR that comprises the amino acid sequence of the VH chain of MF3755 as depicted in FIG. 3, or alternative variable domains that bind EGFR set out herein.

Preferably, the VH chain of the variable domain that binds EGFR comprises the amino acid sequence of VH chain MF3755 as depicted in FIG. 3; or the amino acid sequence of VH chain MF3755 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5816 as depicted in FIG. 3; or the amino acid sequence of VH chain MF5816 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.

Preferably, the variable domain that binds LGR5 binds an epitope that is located within amino acid residues 21-118 of the human LGR5 sequence depicted in FIG. 1. Preferably, the amino acid residues at positions 43, 44, 46, 67, 90, and 91 of human LGR5 are involved in the binding of the LGR5 binding variable domain to LGR5. Preferably, the LGR5 binding variable domain binds less to an LGR5 protein comprising one or more of the amino acid residue variations selected from 43A, 44A, 46A, 67A, 90A, and 91A.

Preferably, the variable domain that binds EGFR binds an epitope that is located within amino acid residues 420-480 of the human EGFR sequence depicted in FIG. 2. Preferably, the amino acid residues at positions I462, G465, K489, I491, N493 and C499 of human EGFR are involved in the binding of the EGFR binding variable domain to EGFR. Preferably, the EGFR binding variable domain binds less to an EGFR protein comprising one or more of the amino acid residue substitutions selected from I462A, G465A, K489A, I491A, N493A and C499A.

Preferably, the antibody is ADCC enhanced. Preferably, the antibody is afucosylated. Preferably, the subject being administered the antibody of the present disclosure has an immune system that allows engaging of the Fc region of the antibody of the present invention. More preferably, said subject comprises FcγRIIIa (CD16+) and/or FcγRIIa (CD32+) immune effector cells to engage the Fc region of the antibody of the present invention. The immune effector cells are preferably Natural Killer cells (NK cells), macrophages or neutrophils comprising said Fc receptors.

### BRIEF DESCRIPTION OF THE DRAWINGS

FIG. 1 Human LGR5 sequence; Sequence ID NO: 1.

FIG. 2 Human EGFR sequence; Sequence ID NO: 2.

FIG. 3 a). Amino acid sequences of heavy chain variable regions (Sequence ID Nos: 3-15) that together with a common light chain variable region such as the variable region of the human kappa light chain IgVκ1 39*01/IGJκ1*01 form a variable domain that binds LGR5 and EGFR. The CDR and framework regions are indicated in FIG. 3b. Respective DNA sequence are indicated in FIG. 3c.

FIG. 4 a). Amino acid sequence of a common light chain amino acid sequence. b) Common light chain variable region DNA sequence and translation (IGKV1-39/jk1). c) Light chain constant region DNA sequence and translation. d) V-region IGKV1-39A; e) CDR1, CDR2 and CDR3 of a common light chain according to IMGT numbering.

FIG. 5. IgG heavy chains for the generation of bispecific molecules. a) CH1 region DNA sequence and translation. b) Hinge region DNA sequence and translation. c) CH2 region DNA sequence and translation. d) CH3 domain containing variations L351K and T366K (KK) DNA sequence and translation. e) CH3 domain containing variations L351D and L368E (DE) DNA sequence and translation. Residue positions are according to EU numbering.

FIG. 6. Data showing mean tumor volume in six head and neck PDX models treated with control and antibody targeting EGFR and LGR5 with relevant error bars based on two-sided testing. Both antibody and control were dosed once a week for six weeks indicated by the grey zone.

### DETAILED DESCRIPTION OF THE DISCLOSED EMBODIMENTS

In order that the present description may be more readily understood, certain terms are first defined. Additional definitions are set forth throughout the detailed description. Unless separately defined herein, all technical and scientific terms used herein have the same meaning as commonly understood by one of ordinary skill in the art, and conventional methods of immunology, protein chemistry, biochemistry, recombinant DNA techniques and pharmacology are employed. As used herein, the singular forms “a”, “an” and “the” include plural referents. Use of the term “comprising” “having” “including” as well as other forms, such as “comprise”, “comprises”, “comprised”, “has”, “have”, “had”, “include”, “includes”, and “included”, is not limiting.

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units-each with two heavy chains and two light chains. An antibody according to the present invention is not limited to any particular format or method of producing it.

A “bispecific antibody” is an antibody as described herein wherein one domain of the antibody binds to a first antigen whereas a second domain of the antibody binds to a second antigen, wherein said first and second antigens are not identical, or where one domain binds a first epitope on an antigen, whereas a second domain binds to a second epitope on the antigen. The term “bispecific antibody” also encompasses antibodies wherein one heavy chain variable region/light chain variable region (VH/VL) combination binds a first antigen or epitope on an antigen and a second VH/VL combination that binds a second antigen or epitope on the antigen. The term further includes antibodies wherein VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. The light chain of the present invention can also be a light chain as specified herein, having from 0 to 10, preferably from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein variations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the antigen binding specificity of the IgG.

A “derivative of an antibody” is a protein that but for the CDR regions deviates from the amino acid sequence of a natural antibody in at most 20 amino acids. A derivative of an antibody as disclosed herein is an antibody that deviates from said amino acid sequence in at most 20 amino acids.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Advance® 11.5.2 software using the default settings, which employ a modified ClustalW algorithm (Thompson, J. D., Higgins, D. G., and Gibson T. J., (1994) Nuc. Acid Res. 22(22): 4673-4680), the swgapdnamt score matrix, a gap opening penalty of 15 and a gap extension penalty of 6.66. Amino acid sequences are aligned with the AlignX application of the Vector NTI Advance® 11.5.2 software using default settings, which employ a modified ClustalW algorithm (Thompson, J. D., Higgins, D. G., and Gibson T. J., (1994) Nuc. Acid Res. 22(22): 4673-4680), the blosum62mt2 score matrix, a gap opening penalty of 10 and a gap extension penalty of 0.1.

As an antibody typically recognizes an epitope of an antigen, and such an epitope may be present in other compounds as well, antibodies according to the present invention that “specifically recognize” an antigen, for example, EGFR or LGR5, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer). Preferably, the subject is a human subject.

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer.

Effective treatment may, for example, decrease in tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of an EGFR/LGR5 antibody to affect a decrease in a cancer (for example a decrease in the number of cancer cells); slowing of progression of a cancer, or prevent regrowth or recurrence of the cancer.

The term “flat dose” herein refers to a dosing regimen wherein a subject is administered with a fixed amount of a therapeutic substance over multiple administrations, independent of body weight of the subject. Flat dosing is typically abbreviated with qnw, wherein n is an integer indicating the interval and w is week. For instance, a q2w flat dose administration regimen of 1500 mg antibody means a fixed amount of 1500 mg antibody is administered each 2 weeks. Herein, the therapeutic substance is preferably an antibody binding EGFR and LGR5 that is administered with a q2w dosing regimen of 1500 mg.

The flat dose may be premedicated, meaning medication is administered to the subject prior to being administered the antibody of the present invention. Preferably, the flat dose of 1500 mg antibody is premedicated with an antihistamine, pain reducing medication, fever reducing medication and/or anti-inflammatory medication.

The present disclosure provides an antibody or functional part, derivative and/or analogue thereof that comprises a variable domain that binds an extracellular part of EGFR and a variable domain that binds an extracellular part of LGR5 for use in the treatment of cancer. The words cancer and tumor are used herein and typically both refer to cancer, unless otherwise specifically stated.

Epidermal growth factor (EGF) receptor (EGFR, ErbB1, or HER1) is a member of a family of four receptor tyrosine kinases (RTKs), named Her- or cErbB-1, -2, -3 and -4. EGFR is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. A major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian, and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is GenBank NM_005228.3. This accession number is primarily given to provide a further method of identification of EGFR protein as a target, the actual sequence of the EGFR protein bound by an antibody may vary, for instance because of a mutation in the encoding gene such as those occurring in some cancers or the like.

Where reference herein is made to EGFR, the reference refers to human EGFR unless otherwise stated. The variable domain antigen-binding site that binds EGFR, binds EGFR and a variety of variants thereof such as those expressed on some EGFR positive tumors.

The term “LGR” refers to the family of proteins known as Leucine-rich repeat-containing G-protein coupled receptors. Several members of the family are known to be involved in the WNT signaling pathway, of note LGR4; LGR5 and LGR6.

LGR5 is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT 029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

Cancers that are known collectively as head and neck cancers usually begin in the squamous cells that line the moist, mucosal surfaces inside the head and neck, such as inside the mouth, the nose, and the throat. These squamous cell cancers are often referred to as squamous cell carcinomas of the head and neck. Although rare, head and neck cancers can also occur in the salivary glands. In particular, the head and neck cancer may occur in the oral cavity. This includes the lips, the front two-thirds of the tongue, the gums, the lining inside the cheeks and lips, the floor of the mouth under the tongue, the hard palate, and the small area of the gum behind the wisdom teeth.

Thus, in particular, the head and neck cancer includes nasopharyngeal cancer, laryngeal cancer. hypopharyngeal cancer, nasal cavity cancer, paranasal sinus cancer, oral cancer, oropharyngeal cancer or salivary gland cancer. More in particular, the present invention relates to treatment of a cancer comprising a squamous cell head and neck cancer located in the oropharynx.

In some disclosures, the cancer expresses LGR5 and/or EGFR. As used herein, a cancer expresses LGR5 if the cancer comprises cells that express LGR5. A cell which expresses LGR5 comprises detectable levels of RNA that codes for LGR5. As used herein, a cancer expresses EGFR if the cancer comprises cells that express EGFR. A cell which expresses EGFR comprises detectable levels of RNA that codes for EGFR. Expression can often also be detected by incubating the cell with an antibody that binds to LGR5 or EGFR. However, some cells do not express the protein high enough for such an antibody test. In such cases mRNA or other forms of nucleic acid sequence detection is preferred. Preferably, EGFR protein expression is detected and LGR5 mRNA expression is detected. Preferably, EGFR and LGR5 detection is by Tissue MicroArray (TMA) staining. LGR5 expression is preferably determined using In-Situ Hybridization (ISH). Thus preferably, the cancer is ISH positive for LGR5. ISH positive preferably means that expression is characterized by an H-score of 1 or more. EGFR expression is preferably determined using immunohistochemistry (IHC). Thus preferably, the cancer is IHC positive for EGFR. Preferably, the cancer has an EGFR IHC score of 0, 1+, 2+ or 3+, more preferably 1+, 2+ or 3+even more preferably 2+ or 3+. The techniques for detection and scoring on the basis thereof, by TMA, ISH and IHC are each well known in the art to the person of ordinary skill and typically commercially available as a standard kit. Preferably, the EGFR score is determined using a commercially available EGFR detection kit, such as the EGFR pharmDx™ kit for a Dako autostainer (Agilent). Quantifying mRNA levels using ISH and expressing such on the basis of an H-score, such as for LGR5, can be performed using commercially available kits, such as the RNAscope R kit from Advanced Cell Diagnostics (Hayward, CA, USA) on a staining platform such as the BondRx platform (Leica, Wetzlar, Germany). Typically, the ISH H-score ranges from 0-400. Alternatively, LGR5 and EGFR expression is determined by RNA sequencing (RNAseq).

The subject may have not previously been treated with an anti-EGFR agent. More preferably, the subject has not been treated with an antibody targeting EGFR, most preferably the subject has not been treated with cetuximab. Such a subject is also referred to as a cetuximab-naïve or anti-EGFR-naïve subject.

Also, the subject may have been previously treated with one or more lines of standard approved therapy or standard of care. Although surgery or radiation therapy may be preferred for most patients with early or localized disease, and may be considered for locally advanced disease, it may not be possible to apply to all patients, for instance due to the anatomical location of the cancer. Standard approved therapy or standard of care herein preferably includes treatment by administration of a chemotherapeutic agent, preferably one or more of a platinum-based compound (e.g. cisplatin, carboplatin), an antineoplastic compound (e.g. methotrexate), a fluoropyrimidine (e.g. fluorouracil, 5-FU, capecitabine), a taxane (e.g. docetaxel or paclitaxel) a nucleoside analogue (e.g. gemcitabine) or any combination thereof.

Cancers, such as head and neck cancer, can be related to the presence of mutations. Such mutations include mutations in known oncogenes such as PIK3CA, KRAS, BRAF, HRAS, MAP2K1 and NOTCH1. Oncogenic mutations are generally described as activating mutations or mutations which result in new functions. Another type of cancer mutation involves tumor suppressor genes, such as TP53, MLH1, CDKN2A, and PTEN. Mutations in tumor suppressor genes are generally inactivating.

Preferably, the cancer has a mutation in one or more EGFR signaling pathway genes. Preferably, the mutation is present in a gene the expression product of which is active downstream of EGFR in the EGFR signaling pathway. More preferably, the cancer has a mutation in one or more EGFR signaling pathway genes which is not active downstream of EGRF.

Preferably, the cancer has a mutation in a gene, and its encoded protein product, selected from AKT1, KRAS, MAP2K1, NRAS, HRAS, PIK3CA, PTEN and EGFR. More preferably, the cancer has a mutation in a gene coding for HRAS and/or PLCG2.

The mutation in the HRAS gene is preferably a missense mutation, a somatic mutation and/or an oncogenic driver mutation. More preferably, HRAS comprises mutation G12S in its protein sequence, or a G>A missense mutation leading to a G>S amino acid change, more preferably missense mutation G34A in the coding sequence (CDS) of the respective GGC codon of the HRAS gene. Preferably, the cancer is oral squamous cell carcinoma or squamous cell carcinoma of the buccal mucosa and comprises missense mutation G12S in the gene coding for HRAS.

The mutation in the PLCG2 gene is preferably mutation R956H, or a G>A missense mutation leading to an R>H amino acid change, or missense mutation G2867A in the coding sequence (CDS) of codon CGC of the PLCG2 gene.

The cancer may have a mutation in the gene coding for MAP2K1. The mutation in the MAP2K1 gene is preferably a missense mutation, a somatic mutation and/or an oncogenic driver mutation. More preferably, MAP2K1 comprises mutation L375R in its protein sequence, or a T>G missense mutation leading to an L>R amino acid change, more preferably missense mutation T1124G in the coding sequence (CDS) of the respective CTC codon in the gene coding for MAP2K1.

Preferably, the cancer does not have a mutation in the gene coding for PIK3C2B and/or PTPN11. Preferably, the mutation in PIK3C2B comprises mutation E1169K in its protein sequence, or a G>A missense mutation leading to an E>K amino acid change, more preferably missense mutation G3505A in the coding sequence (CDS) of the respective GAG codon of the gene coding for PIK3C2B. Preferably, the mutation in PTPN11 comprises mutation G39E in its protein sequence, or a G>A missense mutation leading to an G>E amino acid change, more preferably missense mutation G116A in the coding sequence (CDS) of the respective GGA codon of the gene coding for PTPN11.

Notch 1 (HGNC ID 7881; NOTCH1), also known as AOS5, hN1, AOVD1 and TAN1, is a gene that encodes a transmembrane protein that functions in multiple developmental processes and the interactions between adjacent cells. The transmembrane protein also functions as a receptor for membrane bound ligands. Fusions, missense mutations, nonsense mutations, silent mutations, frameshift deletions and insertions, and in-frame deletions and insertions are observed in cancers such as esophageal cancer, hematopoietic and lymphoid cancers, and stomach cancer. NOTCH1 is altered in 4.48% of all cancers with colon adenocarcinoma, lung adenocarcinoma, breast invasive ductal carcinoma, endometrial endometrioid adenocarcinoma, and skin squamous cell carcinoma having the greatest prevalence of alterations. In head and neck squamous cell carcinoma, NOTCH1 is altered in about 16% of patients (The AACR Project GENIE Consortium. Cancer Discovery. 2017; 7(8):818-831).

TP53 (HGNC ID 11998) encodes a transcription factor that regulates a number of activities include stress response and cell proliferation. Mutations in TP53 are associated with various cancers and are estimated to occur in more than 50% of human cancers, including gastric and esophageal cancer. In particular, the TP53 R248Q mutation was shown to be associated with cancer, including gastric and esophageal cancer (Pitolli et al. Int. J. Mol. Sci. 2019 20:6241). Nonsense mutations at positions R196 and R342 have been identified in a number of tumors such as from breast and esophagus; and ovary, prostate, breast, pancreas, stomach, colon/rectum, lung, esophagus, bone; respectively (Priestly et al. Nature 2019 575:210-216). In some embodiments, the therapeutic compounds disclosed herein are useful for treating a cancer having a TP53 mutation, in particular a mutation that results in reduced TP53 expression or activity.

MLH1 (HGNC ID 7127; MutL homolog 1) encodes a protein involved in DNA mismatch repair and is a known tumor suppressor gene. Mutations in MLH1 are associated with various cancers including gastrointestinal cancer. Low levels of MLH1 are also associated with esophageal cancer patients having a family history of esophageal cancer (Chang et al. Oncol Lett. 2015 9:430-436) and MLH1 is mutated in 1.39% of malignant esophageal neoplasm patients (The AACR Project GENIE Consortium. AACR Project GENIE: powering precision medicine through an international consortium. Cancer Discovery. 2017; 7(8):818-831. Dataset Version 6). In particular, the MLHI V384D mutation was shown to be associated with cancers, e.g., colorectal cancer (Ohsawa et al. Molecular Medicine Reports 2009 2:887-891). In some embodiments, the therapeutic compounds disclosed herein are useful for treating a cancer having a MLH1 mutation, in particular a mutation which results in reduced MLH1 expression or activity.

PIK3CA (HGCN: 8975, phosphatidylinositol-4,5-bisphosphate 3-kinase catalytic subunit alpha) encodes the 110 kDa catalytic subunit of PI3K (phosphatidylinositol 3-kinase). Mutations in PIK3CA are associated with various cancers include gastrointestinal cancer. As reported by the American Association for Cancer Research, PIK3CA is mutated in 12.75% of malignant solid tumor patients. In particular, the PIK3CA H1047R mutation is present in 2.91% of all malignant solid tumor patients and the PIK3CA E545K present in 2.55% of all malignant solid tumor patients (see, The AACR Project GENIE Consortium. AACR Project GENIE: powering precision medicine through an international consortium. Cancer Discovery. 2017; 7(8):818-831. Dataset Version 6.) In some embodiments, the therapeutic compounds disclosed herein are useful for treating a cancer having a PIK3CA mutation, in particular an oncogenic mutation in PIK2CA.

PIK3C2B (HGNC: 8972, Phosphatidylinositol-4-Phosphate 3-Kinase Catalytic Subunit Type 2 Beta), encodes a protein which belongs to the phosphoinositide 3-kinase (PI3K) family. PI3-kinases play roles in signaling pathways involved in cell proliferation, oncogenic transformation, cell survival, cell migration, and intracellular protein trafficking. This protein contains a lipid kinase catalytic domain as well as a C-terminal C2 domain, a characteristic of class II PI3-kinases. C2 domains act as calcium-dependent phospholipid binding motifs that mediate translocation of proteins to membranes, and may also mediate protein-protein interactions.

CDKN2A (HGNC ID 1787; Cyclin-dependent kinase inhibitor 2A) encodes a protein that inhibits CDK4 and ARF. As reported by the American Association for Cancer Research, CDKN2A is mutated in 22.21% of esophageal carcinoma patients. 28.7% of esophageal squamous cell carcinoma patients, and 6.08% of gastric adenocarcinoma patients. In particular, the CDKN2A W110Ter mutation is present in around 0.11% of cancer patients. (The AACR Project GENIE Consortium. AACR Project GENIE: powering precision medicine through an international consortium. Cancer Discovery. 2017; 7(8):818-831. Dataset Version 6). In some embodiments, the therapeutic compounds disclosed herein are useful for treating a cancer having a CDKN2A mutation, in particular a mutation which results in reduced CDKN2A expression or activity.

PTEN (HGNC ID 9588; phosphatase and tensin homolog) encodes for a phosphatidylinositol 3,4,5-trisphosphate 3-phosphatase. As reported by the American Association for Cancer Research, PTEN is mutated in 6.28% of cancer patients, 3.41% of gastric adenocarcinoma patients, 2.37% of esophageal carcinoma patients, and in 2.22% of esophageal adenocarcinoma patients. In particular, the PTEN R130Ter mutation (wherein Ter refers to a termination/stop codon) is present in 0.21% of all colorectal carcinoma patients (The AACR Project GENIE Consortium. AACR Project GENIE: powering precision medicine through an international consortium. Cancer Discovery. 2017; 7(8):818-831. Dataset Version 6). In some embodiments, the therapeutic compounds disclosed herein are useful for treating a cancer having a PTEN mutation, in particular a mutation which results in reduced PTEN expression or activity.

BRAF (HGNC ID: 1097) encodes serine/threonine-protein kinase B-Raf, which is involved in growth signaling. As reported by the American Association for Cancer Research, BRAF is mutated in 1.91% of gastric carcinoma patients and in in 1.93% of gastric adenocarcinoma patients. In particular, the BRAF V600E mutation is present in 2.72% of cancer patients (see, The AACR Project GENIE Consortium. AACR Project GENIE: powering precision medicine through an international consortium. Cancer Discovery. 2017; 7(8):818-831. Dataset Version 6.). In some embodiments, the therapeutic compounds disclosed herein are useful for treating a cancer having a BRAF mutation, in particular an oncogenic mutation in BRAF.

KRAS (HGNC ID 6407; Kirsten RAt Sarcoma) encodes a protein that is party of the RAS/MAPK pathway. As reported by the American Association for Cancer Research, KRAS is mutated in 14.7% of malignant solid tumor patients with KRAS G12C present in 2.28% of all malignant solid tumor patients (see, The AACR Project GENIE Consortium. AACR Project GENIE: powering precision medicine through an international consortium. Cancer Discovery. 2017; 7(8):818-831. Dataset Version 6.). In some embodiments, the therapeutic compounds disclosed herein are useful for treating a cancer having a KRAS mutation, in particular an oncogenic mutation in KRAS.

The HRAS (HGNC ID: 5173) gene product is involved in the activation of Ras protein signal transduction. Ras proteins bind GDP/GTP and possess intrinsic GTPase activity. Somatic mutations in the HRAS proto-oncogene have been shown to be implicated in bladder cancer, thyroid, salivary duct carcinoma, epithelial-myoepithelial carcinoma and kidney cancers (Chiosea et al., in Am. J. of Surg. Path. 39 (6): 744-52: Chiosea et al., in Head and Neck Path. 2014. 8 (2): 146-50). In some embodiments, the therapeutic compounds disclosed herein are useful for treating a cancer having an HRAS mutation, in particular an oncogenic mutation in HRAS, more preferably HRAS mutation G12S. The cancer in particular is HNSCC of the oral cavity or buccal mucosa.

MAP2K1 (HGNC ID: 6840) belongs to the group of mitogen-activated protein kinase kinases. It is active in MAP kinase signaling and encodes for the protein dual specificity mitogen-activated protein kinase kinase 1. As part of the MAP kinase pathway, MAP2K1 is involved in many cellular processes, including cell proliferation, differentiation, and transcriptional regulation. MAP2K1 is altered in 1.05% of all cancers with cutaneous melanoma, lung adenocarcinoma, colon adenocarcinoma, melanoma, and breast invasive ductal carcinoma having the greatest prevalence of alterations (The AACR Project GENIE Consortium. Cancer Discovery. 2017; 7(8):818-831. Dataset Version 8). In some embodiments, the therapeutic compounds disclosed herein are useful for treating a cancer having a MAP2K1 mutation, in particular MAP2K1 mutation L375R.

UGT1A1 (HGNC ID 12530; uridine diphosphateglucuronosyl transferase 1A1) and UGT1A8 (uridine diphosphateglucuronosyl transferase 1A8) encode enzymes of the glucuronidation pathway. Several polymorphisms which reduce enzyme activity are known to affect the metabolism and effect of irinotecan. For example, the UGT1A1*6 allele (G71R polymorphism) having an allele frequency of around 0.13% in Chinese, Korean, and Japanese populations and the

UGT1A1*28 allele (dinucleotide repeat polymorphism in the TATA sequence of the promoter region) are risk factors for irinotecan induced neutropenia. In some embodiments, the therapeutic compounds disclosed herein are useful for treating a cancer having a UGT1A1 and/or UGT1A8 mutation, in particular a mutation that results in reduced expression or activity of UGT1A1 and/or UGT1A8.

In a present disclosure, the head and neck cancer preferably comprises one or more genetic mutations as present in head and neck model HN2167, HN2590, HN2579, HN5124, HN3164, HN3642, HN3411 and/or HN5125 (see Table 1), more preferably one or more genetic mutations as present in head and neck model HN2167, HN2590, HN2579, HN5124, HN3642, HN3411 and/or HN5125. The mutations are preferably somatic mutations, missense mutations, frame-shift mutations, deletions or any combination thereof.

In a present disclosure, the head and neck cancer has one or more mutations in the LGR5 and/or EGFR pathway present in models selected from the group consisting of HN5124, HN5125, HN2579, HN2590, HN2167, HN3642 and HN3164 (see Table 1).

In a present disclosure, the head and neck cancer preferably comprises one or more oncogenic mutations as present in head and neck model HN2167, HN2590, HN2579, HN5124, HN3164, HN3642, HN3411 and/or HN5125 (see Table 1), more preferably one or more genetic mutations as present in head and neck model HN2167, HN2590, HN2579, HN5124, HN3642, HN3411 and/or HN5125. The mutations are preferably somatic mutations, missense mutations, frame-shift mutations, deletions or any combination thereof.

Preferably, the one or more mutations in head and neck cancer, in particular laryngeal cancer, or model HN2167, are selected from the group consisting of CDKN2A (HGNC: 1787), CREBBP (HGNC: 2348), CUL1 (HGNC: 2551), EPHA3 (HGNC: 3387), EXT1 (HGNC: 3512), FAT2 (HGNC: 3596), FOXP1 (HGNC: 3823), HIST1H3B (HGNC: 4776), HSP90AB1 (HGNC: 5258), IKZF3 (HGNC: 13178), IL6ST (HGNC: 6021), INHBA (HGNC: 6066), LMO1 (HGNC: 6641), LPP (HGNC: 6679), MSR1 (HGNC: 7376), NBN (HGNC: 7652), RAD54B (HGNC: 17228), RGS3 (HGNC: 9999), TAOK1 (HGNC: 29259), TP53 (HGNC: 11998) and WNK1 (HGNC: 14540). More preferably, the one or more mutations in head and neck cancer, in particular squamous cell laryngeal carcinoma, comprise CDKN2A, CREPPB, CUL1 and/or TP53. The mutations are preferably somatic mutations, missense mutations, frame-shift mutations, deletions or any combination thereof. CDKN2A preferably comprises a deletion and/or frame-shift mutation, in particular a deletion of amino acids RAGAR at position 99-103 of the CDKN2A protein, more in particular a deletion of nucleic acids GGGCCGGGGCGCGG at position 296-309 of the CDKN2A coding sequence. CREPPB preferably comprises mutation R1446C, or a C>T missense mutation leading to a R>C amino acid change, or missense mutation C4336T in the coding sequence (CDS) of codon CGC of the CREPPB gene. CUL1 preferably comprises mutation D483N, or a G>A missense mutation leading to a D>N amino acid change, or missense mutation G1447A in the coding sequence (CDS) of codon GAT of the CUL1 gene. TP53 preferably comprises mutation R273C, or a C>T missense mutation leading to an R>C amino acid change, or missense mutation C817T in the coding sequence (CDS) of codon CGT of the TP53 gene.

Preferably, the one or more mutations in head and neck cancer, in particular squamous cell carcinoma of the tongue, or model HN2590, are selected from the group consisting of AHR (HGNC: 348), ALK (HGNC: 427), ATP6AP2 (HGNC: 18305), CDKN2A (HGNC: 1787), EP300 (HGNC: 3373), FGFR1 (HGNC: 3688), FLT4 (HGNC: 3767), FN1 (HGNC: 3778), HLA-B (HGNC: 4932), IREB2 (HGNC: 6115), MCM8 (HGNC: 16147), PLCG2 (HGNC: 9066), RB1 (HGNC: 9884), THRAP3 (HGNC: 22964), TP53 (HGNC: 11998), WNK1 (HGNC: 14540), YBX1 (HGNC: 8014) and ZNF638 (HGNC: 17894). More preferably, the one or more mutations in head and neck cancer, in particular cancer of the tongue, comprise EP300, PLCG2 and/or TP53. The mutations are preferably somatic mutations, missense mutations, frame-shift mutations, deletions or any combination thereof. EP300 preferably comprises mutation S1730C, or a C>G missense mutation leading to an S>C amino acid change, or missense mutation C5189G in the coding sequence (CDS) of codon TCT of the EP300 gene. PLCG2 preferably comprises mutation R956H, or a G>A missense mutation leading to an R>H amino acid change, or missense mutation G2867A in the coding sequence (CDS) of codon CGC of the PLCG2 gene. TP53 preferably comprises mutation G245S, or a G>A missense mutation leading to a G>S amino acid change, or missense mutation G733A in the coding sequence (CDS) of codon GGC of the TP53 gene.

Preferably, the one or more mutations in head and neck cancer, in particular squamous cell carcinoma of the buccal mucosa, or model HN2579, are selected from the group consisting of DCC (HGNC: 2701), DLC1 (HGNC: 2897), HRAS (HGNC: 5173), LZTS1 (HGNC: 13861), SMARCA4 (HGNC: 11100) and WRN (HGNC: 12791). More preferably, the one or more mutations in head and neck cancer, in particular squamous cell cancer of the buccal mucosa, comprises HRAS. The mutations are preferably somatic mutations, missense mutations, frame-shift mutations, deletions or any combination thereof. HRAS preferably comprises mutation G12S in its protein sequence, or a G>A missense mutation leading to a G>S amino acid change, more preferably missense mutation G34A in the coding sequence (CDS) of codon GGC of the HRAS gene.

Preferably, the one or more mutations in head and neck cancer, in particular squamous cell carcinoma of the head and neck, or model HN5124, are selected from the group consisting of APC (HGNC: 583), ERCC6 (HGNC: 3438), MAD1L1 (HGNC: 6762) and ROS1 (HGNC: 10261). The mutations are preferably somatic mutations, missense mutations, frame-shift mutations, deletions or any combination thereof. APC preferably comprises mutation R2505Q in its protein sequence, or a G>A missense mutation leading to an R>Q change, more preferably missense mutation G7514A in the coding sequence (CDS) of codon CGA of the APC gene.

Preferably, the one or more mutations in head and neck cancer, in particular adenocarcinoma or parotid adenocarcinoma, or model HN3164, are selected from the group consisting of DLC1 (HGNC: 2897), EPHA4 (HGNC: 3388), KIAA1549 (HGNC: 22219), MAP2K1 (HGNC: 6840), MSH3 (HGNC: 7326) and TP53 (HGNC: 11998). The mutations are preferably somatic mutations, missense mutations, frame-shift mutations, deletions or any combination thereof. MAP2K1 preferably comprises mutation L375R in its protein sequence, or a T>G missense mutation leading to a L>R amino acid change, more preferably missense mutation T1124G in the coding sequence (CDS) of codon CTC of the MAP2K1 gene. TP53 preferably comprises mutation Y234C in its protein sequence, or an A>G missense mutation leading to a Y>C amino acid change, more preferably missense mutation A701G in the coding sequence (CDS) of codon TAC of the TP53 gene.

Preferably, the one or more mutations in head and neck cancer, in particular squamous cell carcinoma of the neck, or model HN5125, are selected from the group consisting of ATM (HGNC: 795), ECT2L (HGNC: 21118), HLA-B (HGNC: 4932), ITGA9 (HGNC: 6145), RB1 (HGNC: 9884), RGS3 (HGNC: 9999), SOX17 (HGNC: 18122) and TP53 (HGNC: 11998). The mutations are preferably somatic mutations, missense mutations, frame-shift mutations, deletions or any combination thereof. SOX17 preferably comprises mutation L156P in its protein sequence, or a T>C missense mutation leading to an L>P amino acid change, more preferably missense mutation T467C in the coding sequence (CDS) of codon CTG of the SOX17 gene. TP53 preferably comprises mutation R337C in its protein sequence, or a C>T missense mutation leading to a R>C amino acid change, more preferably missense mutation C1009T in the coding sequence (CDS) of codon CGC of the TP53 gene.

An antibody or a functional part, derivative and/or analogue thereof as described herein comprises a variable domain that binds an extracellular part of the epidermal growth factor (EGF) receptor and a variable domain that binds LGR5. The EGFR is preferably a human EGFR. The LGR5 is preferably a human LGR5. The antibody or a functional part, derivative and/or analogue thereof as described herein comprises a variable domain that binds an extracellular part of a human epidermal growth factor (EGF) receptor and a variable domain that binds a human LGR5.

Preferably the antibody or a functional part, derivative and/or analogue thereof as described herein comprises a variable domain that binds an extracellular part of the epidermal growth factor (EGF) receptor and interferes with the binding of EGF to the receptor and a variable domain that binds LGR5 wherein interaction of the antibody with LGR5 on an LGR5-expressing cell does not block the binding of an Rspondin (RSPO) to LGR5. Methods for determining whether an antibody blocks or does not block the binding of an Rspondin to LGR5 are described in WO2017069528, which is hereby incorporated by reference.

Where herein accession numbers or alternative names of proteins/genes are given, they are primarily given to provide a further method of identification of the mentioned protein as a target, the actual sequence of the target protein bound by an antibody of the invention may vary, for instance because of a mutation and/or alternative splicing in the encoding gene such as those occurring in some cancers or the like. The target protein is bound by the antibody as long as the epitope is present in the protein and the epitope is accessible to the antibody.

An antibody or a functional part, derivative and/or analogue thereof as described herein preferably interferes with the binding of a ligand for EGFR to EGFR. The term “interferes with binding” as used herein means that binding of the antibody or a functional part, derivative and/or analogue thereof to the EGFR competes with the ligand for binding to EGF receptor. The antibody or a functional part, derivative and/or analogue thereof may diminish ligand binding, displace ligand when this is already bound to the EGF receptor or it may, for instance through steric hindrance, at least partially prevent that ligand can bind to the EGF receptor.

An EGFR antibody as mentioned herein preferably inhibits respectively EGFR ligand-induced signaling. measured as ligand-induced growth of BxPC3 cells (ATCC CRL-1687) or BxPC3-luc2 cells (Perkin Elmer 125058) or ligand-induced cell death of A431 cells (ATCC CRL-1555). EGFR can bind a number of ligands and stimulate growth of the mentioned BxPC3 cells or BxPC3-luc2 cells. In the presence of an EGFR ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The ligand-induced growth is preferably measured using saturating amounts of ligand. In a preferred embodiment EGF is used in an amount of 100 ng/ml of culture medium. EGF is preferably the EGF R&D systems, cat. nr. 396-HB and 236-EG (see also WO2017/069628; which is incorporated by reference herein).

An EGFR antibody as mentioned herein preferably inhibits EGFR ligand induced growth of BxPC3 cells (ATCC CRL-1687) or BxPC3-luc2 cells (Perkin Elmer 125058). EGFR can bind a number of ligands and stimulate growth of the mentioned BxPC3 cells or BxPC3-luc2 cells. In the presence of a ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The ligand-induced growth is preferably measured using saturating amounts of ligand. In a preferred embodiment EGF is used in an amount of 100 ng/ml of culture medium. EGF is preferably the EGF of R&D systems, cat. nr. 396-HB and 236-EG (see also WO2017/069628; which is incorporated by reference herein).

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

Whether an antibody as described herein inhibits signaling or inhibits growth in a multispecific format is preferably determined by the method as described herein above using a monospecific monovalent or monospecific bivalent version of the antibody. Such an antibody preferably has binding sites for the receptor of which signaling is to be determined. A monospecific monovalent antibody can have a variable domain with an irrelevant binding specificity such as tetanus toxoid specificity. A preferred antibody is a bivalent monospecific antibody wherein the antigen binding variable domains consist of variable domains that bind the EGF-receptor family member.

In its Biclonics R antibody program, Merus has developed multispecific antibodies that target EGFR and LGR5 (Leucine-rich repeat containing G protein-coupled receptor). The efficacy of such multispecific antibodies has been assessed in vitro and in vivo using patient-derived CRC organoids and mice PDX models, respectively (see, e.g., WO2017/069628; which is incorporated by reference herein). Multispecific antibodies that target EGFR and LGR5 were shown to inhibit tumor growth. The potency of such inhibitory antibodies was shown to be correlated with the levels of LGR5 RNA expression by cells from the cancer. Multispecific antibodies that target EGFR and LGR5 as described in WO2017/069628 are particularly preferred.

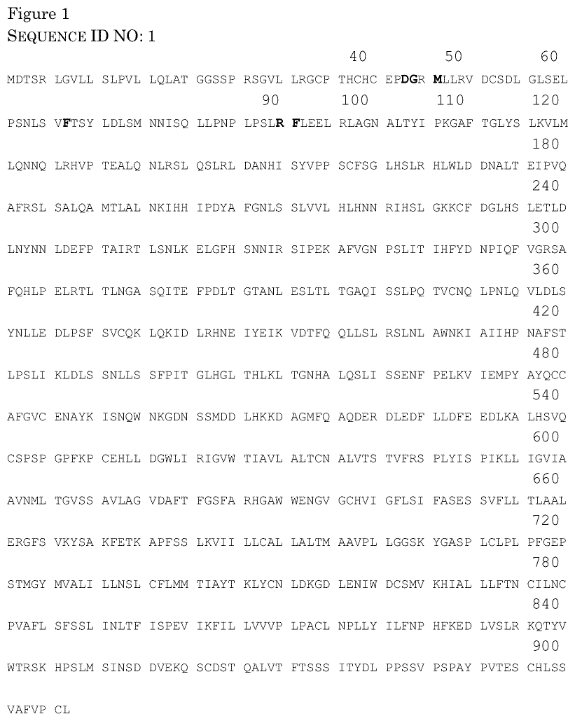

An antibody or a functional part, derivative and/or analogue thereof as described herein comprises a variable domain that binds an extracellular part of LGR5. The variable domain that binds an extracellular part of LGR5 preferably binds an epitope that is located within amino acid residues 21-118 of the sequence of FIG. 1 of which amino acid residues D43; G44, M46, F67, R90, and F91 are involved in binding of the antibody to the epitope.

The LGR5 variable domain is preferably a variable domain wherein one or more of the amino acid residue substitutions in LGR5 of D43A; G44A, M46A, F67A, R90A, and F91A reduces the binding of the variable domain to LGR5.

The epitope on an extracellular part of LGR5 is preferably located within amino acid residues 21-118 of the sequence of FIG. 1. It is preferably an epitope wherein the binding of the LGR5 variable domain to LGR5 is reduced by one or more of the following amino acid residue substitutions D43A; G44A, M46A, F67A, R90A, and F91A in LGR5.

The disclosure further provides an antibody with a variable domain that binds an extracellular part of EGFR and a variable domain that binds an extracellular part of LGR5 wherein the LGR5 variable domain binds an epitope on LGR5 that is located within amino acid residues 21-118 of the sequence of FIG. 1

The epitope on LGR5 is preferably a conformational epitope. The epitope is preferably located within amino acid residues 40-95 of the sequence of FIG. 1. The binding of the antibody to LGR5 is preferably reduced with one or more of the following amino acid residue substitutions D43A; G44A, M46A, F67A, R90A, and F91A.

Without being bound by theory it is believed that M46, F67, R90, and F91 of LGR5 as depicted in FIG. 1, are contact residues for a variable domain as indicated herein above, i.e. the antigen-binding site of a variable domain that binds the LGR5 epitope. That amino acid residue substitution D43A and G44A reduces the binding of an antibody can be due to the fact that these are also contact residues, however, it is also possible that these amino acid residue substitutions induce a (slight) modification of the conformation of the part of LGR5 that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In an exemplary method, CHO cells express LGR5 on the cell membrane, or on alanine substitution mutant, preferably a mutant comprising one or more of the substitutions M46A, F67A, R90A, or F91A. A test antibody is contacted with the CHO cells and binding of the antibody to the cells compared. A test antibody binds the epitope if it binds to LGR5 and to a lesser extent to an LGR5 with a M46A, F67A, R90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein and a part that warrants association with the cell membrane of course, when cells are used. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel readily identifies whether the test antibodies exhibit reduced binding to mutants with a M46A, F67A, R90A, or F91A substitution and thus whether the test antibody is an antibody of the invention. Reduced binding to mutants with a M46A, F67A. R90A, or F91A substitution also identifies the epitope to be located within amino acid residues 21-118 of the sequence of FIG. 1. In a preferred embodiment the panel includes a D43A substitution mutant; a G44A substitution mutant of both. The antibody with the VH sequence of the VH of MF5816 exhibits reduced binding to these substitution mutants.

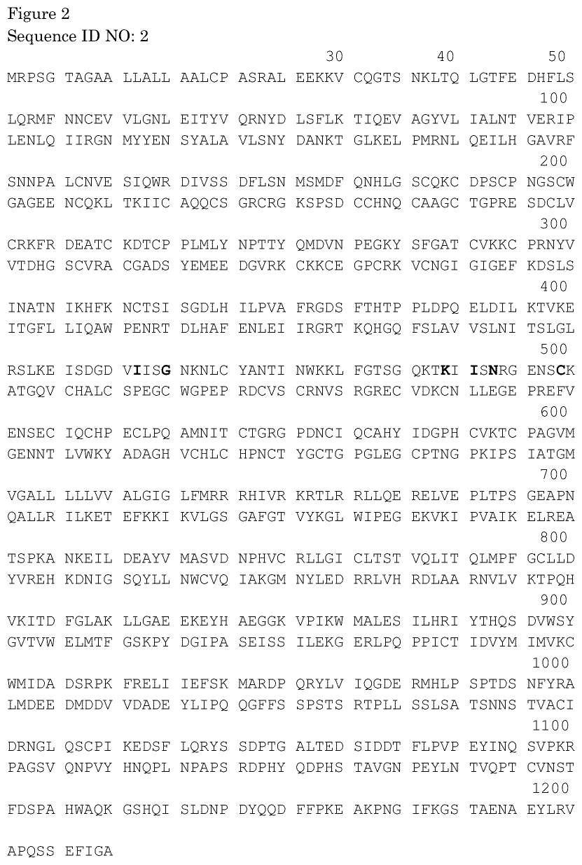

Without being bound by any theory it is believed that amino acid residues I462; G465; K489; I491; N493; and C499 as depicted FIG. 2 are involved in binding an epitope by an antibody comprising a variable domain as indicated herein above. Involvement in binding is preferably determined by observing a reduced binding of the variable domain to an EGFR with one or more of the amino acid residue substitutions selected from 1462A; G465A; K489A; 1491A; N493A; and C499A.

In one aspect, the variable domain that binds an epitope on an extracellular part of human EGFR is a variable domain that binds an epitope that is located within amino acid residues 420-480 of the sequence depicted in FIG. 2. Preferably the binding of the variable domain to EGFR is reduced by one or more of the following amino acid residue substitutions 1462A; G465A; K489A; I491A; N493A; and C499A in EGFR. The binding of the antibody to human EGFR preferably interferes with the binding of EGF to the receptor. The epitope on EGFR is preferably a conformational epitope. In one aspect the epitope is located within amino acid residues 420-480 of the sequence depicted in FIG. 2, preferably within 430-480 of the sequence depicted in FIG. 2; preferably within 438-469 of the sequence depicted in FIG. 2.

Without being bound by theory it is believed that the contact residues of the epitope, i.e. where the variable domain contacts the human EGFR are likely I462; K489; I491; and N493. The amino acid residues G465 and C499 are likely indirectly involved in the binding of the antibody to EGFR.

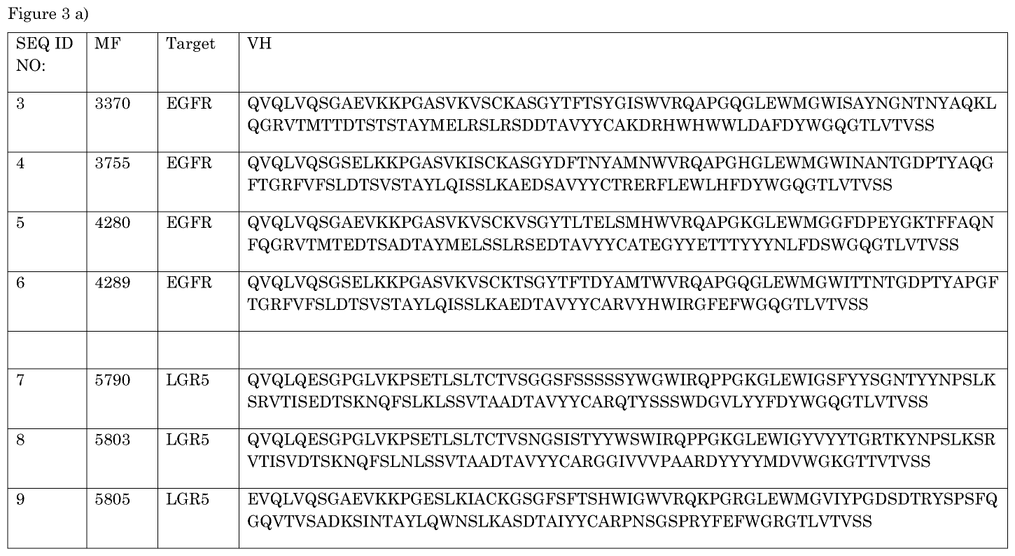

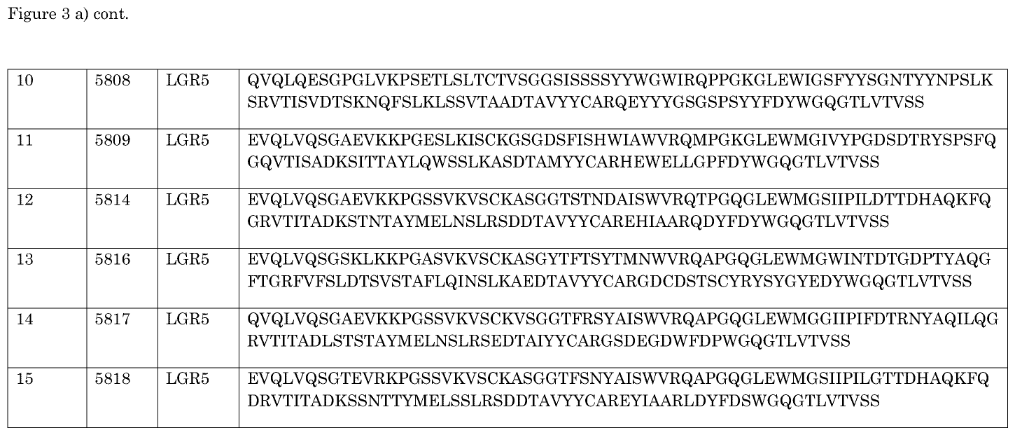

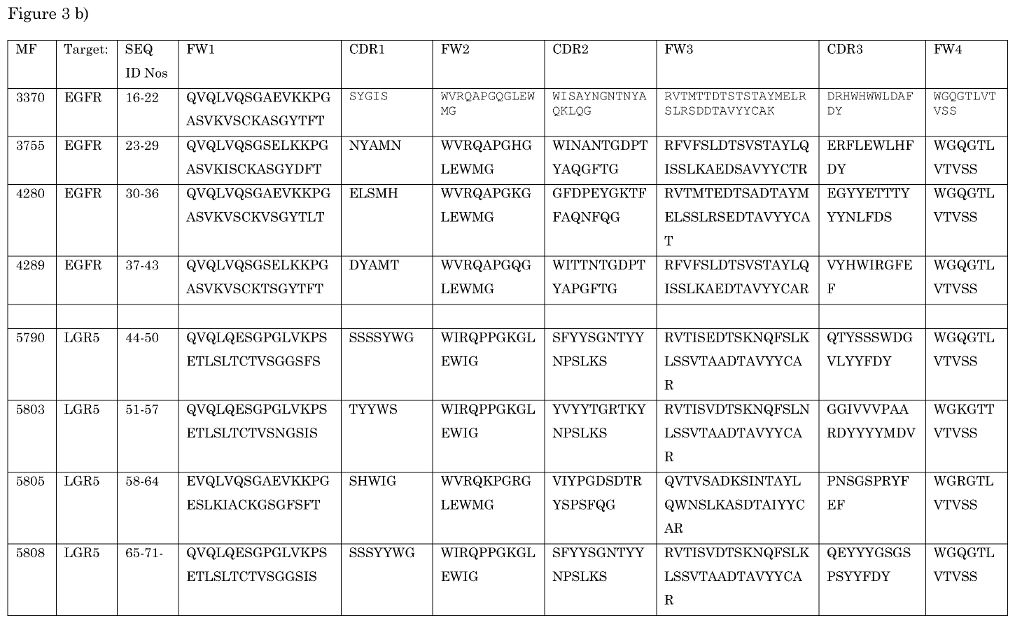

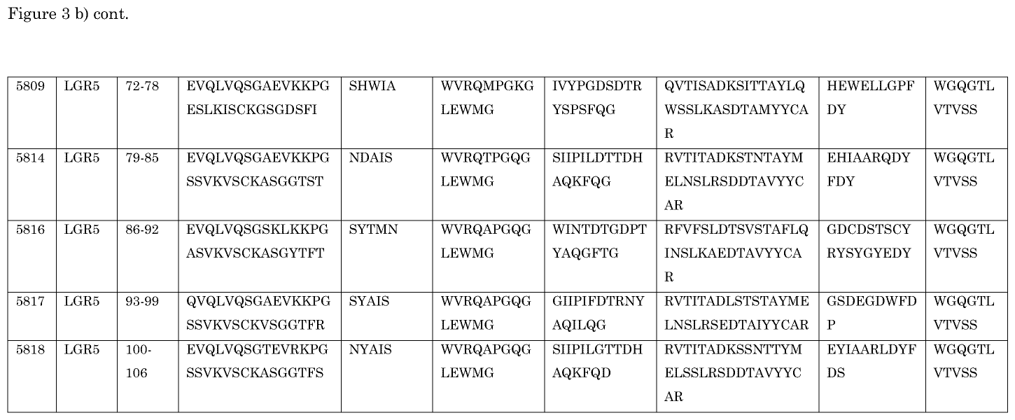

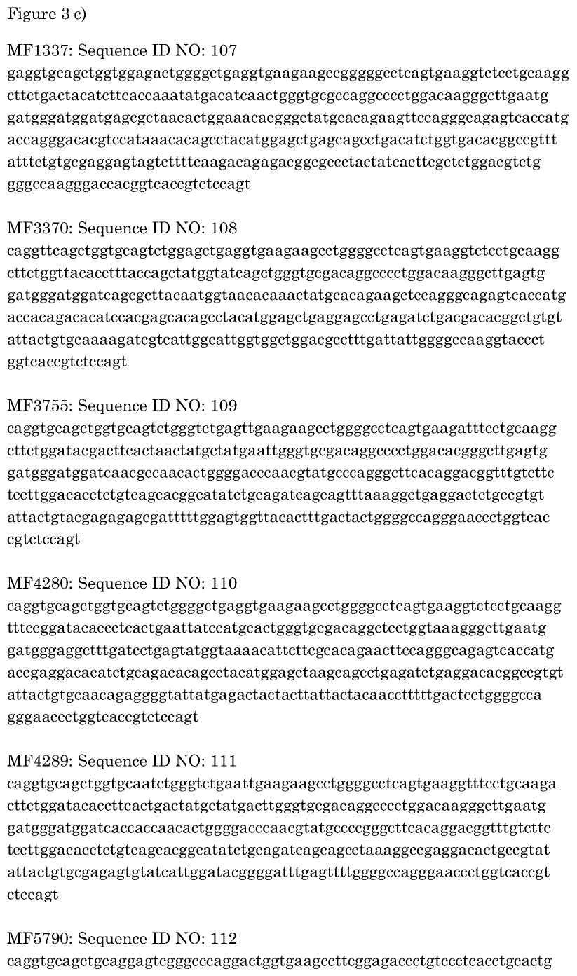

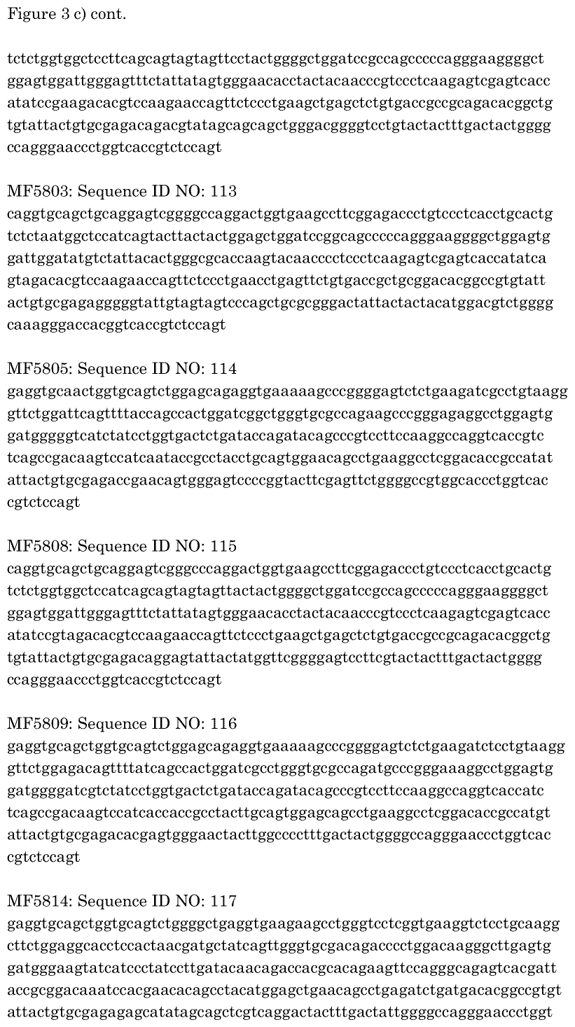

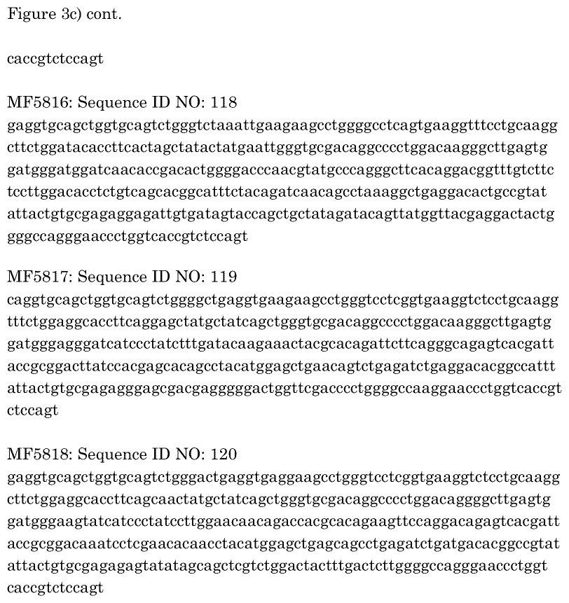

The variable domain that binds human EGFR, is preferably a variable domain with a heavy chain variable region that comprises at least the CDR3 sequence of the VH of MF3755 as depicted in FIG. 3 or a CDR3 sequence that differs in at most three, preferably in at most two, preferably in no more than one amino acid from a CDR3 sequence of the VH of MF3755 as depicted in FIG. 3. The variable domain that binds human EGFR, is preferably a variable domain with a heavy chain variable region that comprises at least the CDR1, CDR2 and CDR3 sequences of the VH of MF3755 as depicted in FIG. 3; or the CDR1, CDR2 and CDR3 sequences of the VH of MF3755 as depicted in FIG. 3 with at most three, preferably at most two, preferably at most one amino acid substitutions.

The variable domain that binds human EGFR, is preferably a variable domain with a heavy chain variable region that comprises the sequence of the VH chain of MF3755 as depicted in FIG. 3; or the amino acid sequence of the VH chain of MF3755 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3755.

In one aspect, the disclosure provides an antibody comprising a variable domain that binds an extracellular part of EGFR and a variable domain that binds an extracellular part of LGR5, wherein a heavy chain variable region of said variable domain comprises at least the CDR3 sequence of an EGFR specific heavy chain variable region selected from the group consisting of MF3370; MF3755; MF4280 or MF4289 as depicted in FIG. 3 or wherein a heavy chain variable region of said variable domain comprises a heavy chain CDR3 sequence that differs in at most three, preferably in at most two, preferably in no more than one amino acid from a CDR3 sequence of a VH selected from the group consisting of MF3370; MF3755; MF4280 or MF4289 as depicted in FIG. 3. Said variable domain preferably comprises a heavy chain variable region comprising at least the CDR3 sequence of MF3370; MF3755; MF4280 or MF4289 as depicted in FIG. 3.

Said variable domain preferably comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 3, or heavy chain variable region comprising at least CDR1, CDR2 and CDR3 sequences that differ in at most three, preferably in at most two, preferably in at most one amino acid from the CDR1, CDR2 and CDR3 sequences of an EGFR specific heavy chain variable region selected from the group consisting of MF3370; MF3755; MF4280 or MF4289 as depicted in FIG. 3. Said variable domain preferably comprises a heavy chain variable region comprising at least the CDR1, CDR2 and CDR3 sequences of MF3370; MF3755; MF4280 or MF4289 as depicted in FIG. 3. A preferred heavy chain variable region is MF3755. Another preferred heavy chain variable region is MF4280.

The antibody comprising a variable domain that binds an extracellular part of EGFR and a variable domain that binds an extracellular part of LGR5, wherein the EGFR binding variable domains has a CDR3, a CDR1, CDR2, and CDR3 and/or a VH sequence as indicated herein above preferably has a variable domain that binds LGR5 that comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 3 or a heavy chain CDR3 sequence that differs in at most three, preferably in at most two, preferably in no more than one amino acid from a CDR3 sequence of a VH selected from the group consisting of MF5790; MF5803; MF5805; MF5808; MF5809; MF5814; MF5816; MF5817; or MF5818 as depicted in FIG. 3. Said variable domain preferably comprises a heavy chain variable region comprising at least the CDR3 sequence of MF5790; MF5803; MF 5805; MF5808; MF5809; MF5814; MF5816; MF5817; or MF5818 as depicted in FIG. 3.

The LGR5 variable domain preferably comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3, or heavy chain CDR1, CDR2 and CDR3 sequences that differ in at most three, preferably in at most two, preferably in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 3. Said variable domain preferably comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 3. Preferred heavy chain variable regions are MF5790; MF5803; MF5814; MF5816; MF5817; or MF5818. Particularly preferred heavy chain variable regions are MF5790; MF5814; MF5816; and MF5818; preferably MF5814, MF5818 and MF5816, heavy chain variable region MF5816 is particularly preferred. Another preferred heavy chain variable region is MF5818.

It has been shown that the antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5818 or one or more CDRs thereof.

VH chains of variable domains that bind EGFR or LGR5 can have one or more amino acid substitutions with respect to the sequence depicted in FIG. 3. A VH chain preferably has an amino acid sequence of an EGFR or LGR5 VH of FIG. 3, having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 3.

CDR sequences can have one or more an amino acid residue substitutions with respect to a CDR sequence in the figures. Such one or more substitutions are for instance made for optimization purposes, preferably in order to improve binding strength or the stability of the antibody. Optimization is for instance performed by mutagenesis procedures where after the stability and/or binding affinity of the resulting antibodies are preferably tested and an improved EGFR specific CDR sequence or LGR5 specific CDR sequence is preferably selected. A skilled person is well capable of generating antibody variants comprising at least one altered CDR sequence according to the invention. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

Preferably, the mentioned at most 15, preferably 1, 2, 3, 4, 5, 6, 7, 8, 9 or 10 and preferably 1, 2, 3, 4 or 5 amino acid substitutions in a VH or VL as specified herein are preferably conservative amino acid substitutions. The amino acid insertions, deletions and substitutions in a VH or VL as specified herein are preferably not present in the CDR3 region. The mentioned amino acid insertions, deletions and substitutions are preferably also not present in the CDR1 and CDR2 regions. The mentioned amino acid insertions, deletions and substitutions are preferably also not present in the FR4 region.

The mentioned at most 15, preferably 1, 2, 3, 4, 5, 6, 7, 8, 9 or 10 and preferably 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

An antibody comprising a variable domain that binds an extracellular part of EGFR and a variable domain that binds an extracellular part of LGR5 preferably comprises
the amino acid sequence of VH chain MF3755 as depicted in FIG. 3; or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5790 as depicted in FIG. 3; or
the amino acid sequence of VH chain MF5790 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 3; or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5803 as depicted in FIG. 3; or
the amino acid sequence of VH chain MF5803 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 3; or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5814 as depicted in FIG. 3; or
the amino acid sequence of VH chain MF5814 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 3; or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5816 as depicted in FIG. 3; or
the amino acid sequence of VH chain MF5816 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 3; or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5817 as depicted in FIG. 3; or
the amino acid sequence of VH chain MF5817 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

An antibody comprising a variable domain that binds an extracellular part of EGFR and a variable domain that binds an extracellular part of LGR5 preferably comprises
the amino acid sequence of VH chain MF3755 as depicted in FIG. 3 or
the amino acid sequence of VH chain MF3755 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
wherein the VH chain of the variable domain that binds LGR5 comprises
the amino acid sequence of VH chain MF5818 as depicted in FIG. 3; or
the amino acid sequence of VH chain MF5818 as depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

Additional variants of the herein described amino acid sequences which retain EGFR or LGR5 binding can be obtained, for example, from phage display libraries which contain the rearranged human IGKVl-39/IGKJl VL region (De Kruif et al. Biotechnol Bioeng. 2010 (106)741-50), and a collection of VH regions incorporating amino acid substitutions into the amino acid sequence of an EGFR or LGR5 VH region disclosed herein, as previously described (e.g., WO2017/069628). Phages encoding Fab regions which bind EGFR or LGR5 may be selected and analyzed by flow cytometry, and sequenced to identify variants with amino acid substitutions, insertions, deletions or additions which retain antigen binding.

The light chain variable regions of the VH/VL EGFR and LGR5 variable domains of the EGFR/LGR5 antibody may be the same or different. In some embodiments, the VL region of the VH/VL EGFR variable domain of the EGFR/LGR5 antibody is similar to the VL region of the VH/VL LGR5 variable domain. In certain embodiments, VL regions in the first and second VH/VL variable domains are identical.

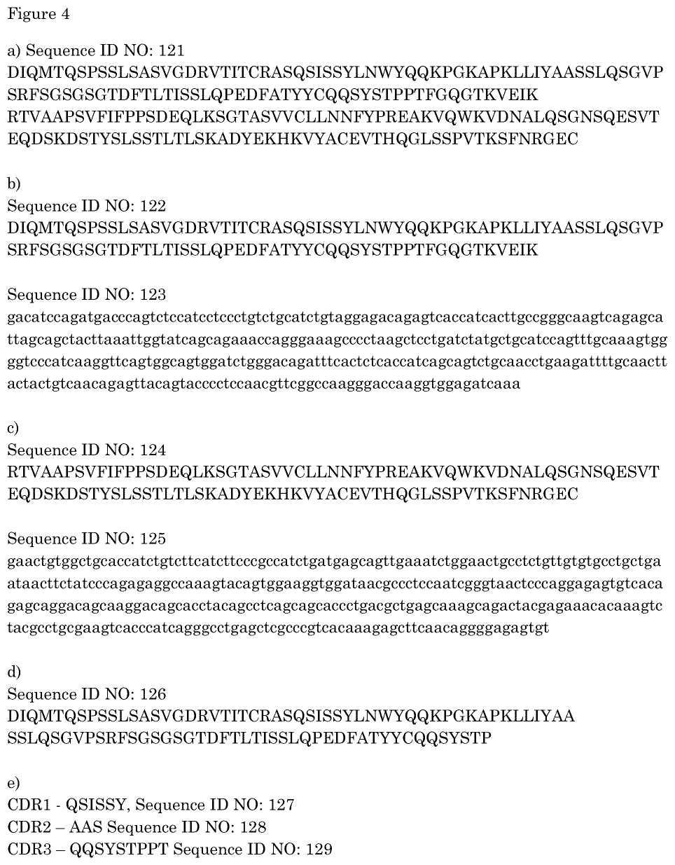

In certain aspects, the light chain variable region of one or both VH/VL variable domains of the EGFR/LGR5 antibody comprises a common light chain variable region. In some aspects, the common light chain variable region of one or both VH/VL variable domains comprises a germline IgVκ1-39 variable region V-segment. In a certain aspect, the light chain variable region of one or both VH/VL variable domains comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 4. The V-region can be combined with one of five J-regions. Preferred J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain embodiments, the light chain variable region of one or both VH/VL variable domains comprises the kappa light chain IgVκ1-39*01/IGJκ1*01 or IgVκ1-39*01/IGJκ1*05 (described in FIG. 4).

In some aspects, the light chain variable region of one or both VH/VL variable domains of the EGFR/LGR5 bispecific antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTP (described in FIG. 4) (i.e., the CDRs of IGKV1-39 according to IMGT). In some aspects, the light chain variable region of one or both VH/VL variable domains of the EGFR/LGR5 antibody comprises an LCDR1 comprising the amino acid sequence QSISSY (described in FIG. 4), an LCDR2 comprising the amino acid sequence AASLQS (described in FIG. 4), and an LCDR3 comprising the amino acid sequence QQSYSTP (described in FIG. 4).

In some aspects, one or both VH/VL variable domains of the EGFR/LGR5 antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in FIG. 4. In some aspects, one or both VH/VL variable domains of the EGFR/LGR5 antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in FIG. 4.

For example, in some aspects, the variable light chain of one or both VH/VL variable domains of the EGFR/LGR5 antibody can have from 0 to 10, preferably from 0 to 5 amino acid insertions, deletions, substitutions, additions or a combination thereof with respect to a sequence in FIG. 4. In some aspects, the light chain variable region of one or both VH/VL variable domains of the EGFR/LGR5 antibody comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other aspects, the light chain variable region of one or both VH/VL variable domains of the EGFR/LGR5 antibody comprises the amino acid sequence of a sequence as depicted in FIG. 4. In certain aspects, both VH/VL variable domains of the EGFR/LGR5 antibody comprise identical VL regions. In one embodiment, the VL of both VH/VL variable domains of the EGFR/LGR5 bispecific antibody comprises the amino acid sequence set forth in FIG. 4. In one aspect, the VL of both VH/VL variable domains of the EGFR/LGR5 bispecific antibody comprises the amino acid sequence set forth in FIG. 4.

The EGFR/LGR5 antibody as described herein is preferably a bispecific antibody having two variable domains, one that binds EGFR and another that binds LGR5 as described herein. EGFR/LGR5 bispecific antibodies for use in the methods disclosed herein can be provided in a number of formats. Many different formats of bispecific antibodies are known in the art, and have been reviewed by Kontermann (Drug Discov Today, 2015 July; 20(7):838-47; MAbs, 2012 March-April; 4(2):182-97) and in Spiess et al., (Alternative molecular formats and therapeutic applications for bispecific antibodies. Mol. Immunol. (2015) http://dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In some aspects, the EGFR/LGR5 bispecific antibodies used in the methods provided herein are generally of the human IgG subclass (e.g., for instance IgG1, IgG2, IgG3, IgG4). In certain aspects, the antibodies are of the human IgG1 subclass. Full length IgG antibodies are preferred because of their favorable half-life and for reasons of low immunogenicity. Accordingly, in certain aspects, the EGFR/LGR5 bispecific antibody is a full length IgG molecule. In an aspect, the EGFR/LGR5 bispecific antibody is a full length IgG1 molecule.

Accordingly, in certain aspects, the EGFR/LGR5 bispecific antibody comprises a fragment crystallizable (Fc). The Fc of the EGFR/LGR5 bispecific antibody is preferably comprised of a human constant region. A constant region or Fc of the EGFR/LGR5 bispecific antibody may contain one or more, preferably not more than 10, preferably not more than 5 amino-acid differences with a constant region of a naturally occurring human antibody. For example, in certain aspects, each Fab-arm of the bispecific antibodies may further include an Fc-region comprising modifications promoting the formation of the bispecific antibody, promoting stability and/or other features described herein.

Bispecific antibodies are typically produced by cells that express nucleic acid(s) encoding the antibody. Accordingly, in some aspects, the bispecific EGFR/LGR5 antibodies disclosed herein are produced by providing a cell comprising one or more nucleic acids that encode the heavy and light chain variable regions and constant regions of the bispecific EGFR/LGR5 antibody. The cell is preferably an animal cell, more preferably a mammal cell, more preferably a primate cell, most preferably a human cell. A suitable cell is any cell capable of comprising and preferably of producing the EGFR/LGR5 bispecific antibody.

Suitable cells for antibody production are known in the art and include a hybridoma cell, a Chinese hamster ovary (CHO) cell, an NS0 cell or a PER-C6 cell. Various institutions and companies have developed cell lines for the large scale production of antibodies, for instance for clinical use. Non-limiting examples of such cell lines are CHO cells, NS0 cells or PER.C6 cells. In a particularly preferred embodiment said cell is a human cell. Preferably a cell is transformed by an adenovirus E1 region or a functional equivalent thereof. A preferred example of such a cell line is the PER.C6 cell line or equivalent thereof. In a particularly preferred embodiment said cell is a CHO cell or a variant thereof. Preferably the variant makes use of a Glutamine synthetase (GS) vector system for expression of an antibody. In one preferred aspect, the cell is a CHO cell.

In some aspects, the cell expresses the different light and heavy chains that make up the EGFR/LGR5 bispecific antibody. In certain aspects, the cell expresses two different heavy chains and at least one light chain. In one preferred embodiment, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1 39/IGKJ1 (huVκ1 39) light chain, previously shown to be able to pair with more than one heavy chain thereby giving rise to antibodies with diverse specificities, which facilitates the generation of bispecific molecules (De Kruif et al. J. Mol. Biol. 2009 (387) 548 58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred aspectthe bispecific antibody of the invention comprises two different immunoglobulin heavy chains with compatible heterodimerization domains. Various compatible heterodimerization domains have been described in the art. The compatible heterodimerization domains are preferably compatible immunoglobulin heavy chain CH3 heterodimerization domains. The art describes various ways in which such hetero-dimerization of heavy chains can be achieved.

One preferred method for producing the EGFR/LGR5 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in one aspect the heavy chain/light chain combination that comprises the variable domain that binds EGFR, comprises a DE variant of the heavy chain. In this embodiment the heavy chain/light chain combination that comprises the variable domain that binds LGR5 comprises a KK variant of the heavy chain.

A candidate EGFR/LGR5 IgG bispecific antibody can be tested for binding using any suitable assay. For example, binding to membrane-expressed EGFR or LGR5 on CHO cells can be assessed by flow cytometry (according to the FACS procedure as previously described in WO2017/069628). In one aspect, the binding of a candidate EGFR/LGR5 bispecific antibody to LGR5 on CHO cells is demonstrated by flow cytometry, performed according to standard procedures known in the art. Binding to the CHO cells is compared with CHO cells that have not been transfected with expression cassettes for EGFR and/or LGR5. The binding of the candidate bispecific IgG1 to EGFR is determined using CHO cells transfected with an EGFR expression construct; a LGR5 monospecific antibody and an EGFR monospecific antibody, as well as an irrelevant IgG1 isotype control mAb are included in the assay as controls (e.g., an antibody which binds LGR5 and another antigen such as tetanus toxin (TT)).

The affinities of the LGR5 and EGFR Fabs of a candidate EGFR/LGR5 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human EGFR (Sino Biological Inc, cat. Nr. 11896-H07H) and human LGR5 protein are run over the sensor surface in a concentration range to measure on- and off-rates. After each cycle, the sensor surface is regenerated by a pulse of HCl and the bsAb is captured again. From the obtained sensorgrams, on- and off-rates and affinity values for binding to human LGR5 and EGFR are determined using the BIAevaluation software, as previously described for CD3 in US 2016/0368988.

An antibody as described herein is typically a bispecific full length antibody, preferably of the human IgG subclass, preferably of the human IgG1 subclass. Such antibodies have good ADCC properties which can, if desired, be enhanced by techniques known in the art, have favorable half-life upon in vivo administration to humans and CH3 engineering technology exists that can provide for modified heavy chains that preferentially form heterodimers over homodimers upon co-expression in clonal cells.

ADCC activity of an antibody can be improved when the antibody itself has a low ADCC activity, by modifying the constant region of the antibody. Another way to improve ADCC activity of an antibody is by enzymatically interfering with the glycosylation pathway resulting in a reduced fucose. Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody as described herein is preferably ADCC enhanced. A bispecific antibody can in one aspect be afucosylated. A bispecific antibody preferably comprises a reduced amount of fucosylation of the N-linked carbohydrate structure in the Fc region, when compared to the same antibody produced in a normal CHO cell.

The antibody that comprises a variable domain that binds an extracellular part of EGFR and a variable domain that binds an extracellular part of LGR5 may further comprise one or more additional variable domains that can bind one or more further targets. The further target is preferably a protein, preferably a membrane protein comprising an extracellular part. A membrane protein as used herein is a cell membrane protein, such as a protein that is in the outer membrane of a cell, the membrane that separates the cell from the outside world. The membrane protein has an extracellular part. A membrane protein is at least on a cell if it contains a transmembrane region that is in the cell membrane of the cell.

Antibodies with more than two variable domains are known in the art. For instance, it is possible to attach an additional variable domain to a constant part of the antibody. An antibody with three or more variable domains is preferably a multivalent multimer antibody as described in PCT/NL2019/050199 which is incorporated by reference herein.

In one aspect the antibody is a bispecific antibody comprising two variable domains, wherein one variable domain binds an extracellular part of EGFR and another variable domain binds an extracellular part of LGR5. The variable domains are preferably variable domains as described herein.

A functional part of an antibody as described herein comprises at least a variable domain that binds an extracellular part of EGFR and a variable domain that binds an extracellular part of LGR5 as described herein. It thus comprises the antigen binding parts of an antibody as described herein and typically contains the variable domains of the antibody. A variable domain of a functional part can be a single chain Fv-fragment or a so-called single domain antibody fragment. A single-domain antibody fragment (sdAb) is an antibody fragment with a single monomeric variable antibody domain. Like a whole antibody, it is able to bind selectively to a specific antigen. With a molecular weight of only 12-15 kDa, single-domain antibody fragments are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Single-domain antibodies by themselves are not much smaller than normal antibodies (being typically 90-100 kDa). Single-domain antibody fragments are mostly engineered from heavy-chain antibodies found in camelids; these are called VHH fragments (Nanobodies®). Some fishes also have heavy-chain only antibodies (IgNAR, ‘immunoglobulin new antigen receptor’), from which single-domain antibody fragments called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Non-limiting examples of such variable domains of antibody parts are VHH, Human Domain Antibodies (dAbs) and Unibodies. Preferred antibody parts or derivatives have at least two variable domains of an antibody or equivalents thereof. Non-limiting examples of such variable domains or equivalents thereof are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

Also provided is an antibody or functional part, derivative and/or analogue thereof as disclosed herein (i.e., the therapeutic compound) and a pharmaceutically acceptable carrier. Such pharmaceutical compositions are useful in the treatment of cancer, in particular for the treatment of head and neck cancer. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent. adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra-articularly and the like) the active compound may be coated in a material to protect the compound from the action of acids and other natural conditions that may inactivate the compound.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage. Also included are solid preparations which are intended for conversion, shortly before use, to liquid preparations for either oral or parenteral administration. Such liquid forms include solutions, suspensions and emulsions.

The therapeutic compound disclosed can be administered according to a suitable dosage, and suitable route (e.g., intravenous, intraperitoneal, intramuscular, intrathecal or subcutaneous). For example, a single bolus may be administered, several divided doses may be administered over time or the dose may be proportionally reduced or increased as indicated by the exigencies of the therapeutic situation. In one embodiment, a subject is administered a single dose of a the antibody or functional part, derivative and/or analogue thereof as disclosed herein. In some embodiments, the therapeutic compound will be administered repeatedly, over a course of treatment. For example, in certain embodiments, multiple (e.g., 2, 3, 4, 5, 6, 7, 8, 9, 10 or more) doses of the therapeutic compound are administered to a subject in need of treatment. In some embodiments, administrations of the therapeutic compound may be done weekly, biweekly or monthly. Preferably, the antibody of the invention is administered on a biweekly basis.

A clinician may utilize preferred dosages as warranted by the condition of the patient being treated. The dose may depend upon a number of factors, including stage of disease, etc. Determining the specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain aspects, the therapeutic compound is administered at a dose of 0.1, 0.3, 1, 2, 3, 4, 5, 6, 7, 8, 9, or 10 mg/kg body weight. In another embodiment, the therapeutic compound is administered at a dose of 0.5, 1, 2, 3, 4, 5, 6, 7, 8, 9, or 10 mg/kg body weight.

In a preferred aspect, the therapeutic compound (i.e., an antibody or functional part, derivative and/or analogue thereof that comprises a variable domain that binds an extracellular part of EGFR and a variable domain that binds an extracellular part of LGR5) is provided to a subject at a dosage of 1500 mg. A flat dosage offers several advantages over body-surface or weight dosing as it reduces preparation time and reduces potential dose calculation mistakes. In some embodiments, the therapeutic compound is provided at a dosage of at least 1100 mg, preferably at a dosage of between 1100 to 2000 mg, more preferably at a dosage of between 1100 to 1800 mg. As is understood by the skilled person, the dosage can be administered over time. For example, the dosage may be administered by IV, for example with a 1-6 hour infusion, preferably a 2-4 hour infusion. In some embodiments, the therapeutic compound is administered once every 2 weeks. In some embodiments, the flat dosages disclosed herein are suitable for use in adults and/or in subjects weighing at least 35 kg. Preferably, the subject is afflicted with head and neck cancer.

The present disclosure provides that a premedication regimen may be used. Such a regimen may be applied to reduce the likelihood or severity of an infusion-related reaction. Preferably, a steroid or corticosteroid (such as dexamethasone) and/or an antihistamine or H1 antagonist (such as dexchlorpheniramine, diphenhydramine, or chlorpheniramine), or medication to reduce production of gastric acid (such as ranitidine) is administered (e.g., orally, intravenously) prior to antibody treatment. Also, medication to reduce, treat or alleviate pain or fever may be premedicated, such by administering paracetamol or the like.

A preferred premedication regimen includes dexamethasone 20 mg (IV), dexchlorpheniramine 5 mg (IV) or diphenhydramine 50 mg (PO) or chlorpheniramine 10 mg (IV) and ranitidine 50 mg (IV) or 150 mg (PO), and paracetamol 1 g (IV) or 650 mg (PO).

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable).

With regard to the frequency of administering the therapeutic compound, one of ordinary skill in the art will be able to determine an appropriate frequency. For example, a clinician can decide to administer the therapeutic compound relatively infrequently (e.g., once every two weeks) and progressively shorten the period between doses as tolerated by the patient. Exemplary lengths of time associated with the course of therapy in accordance with the claimed method include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

The efficacy of the treatment methods provided herein can be assessed using any suitable means. In one embodiment, the clinical efficacy of the treatment is analyzed using cancer cell number reduction as an objective response criterion. Patients, e.g., humans, treated according to the methods disclosed herein preferably experience improvement in at least one sign of cancer. In some embodiments, one or more of the following can occur; the number of cancer cells can be reduced; cancer recurrence is prevented or delayed; one or more of the symptoms associated with cancer can be relieved to some extent. In addition, in vitro assays to determine the T cell mediated target cell lysis. In some embodiments, tumor assessment is based on CT-scan and/or MRI scans, see, e.g., the RECIST 1.1 guidelines (Response Evaluation Criteria in Solid Tumours) (Eisenhauer et al., 2009 Eur J Cancer 45:228-247). Such assessments generally take place every 4-8 weeks after treatment.

In some aspects, the tumor cells are no longer detectable following treatment as described herein. In some embodiments, a subject is in partial or full remission. In certain aspects, a subject has an increased overall survival, median survival rate, and/or progression free survival.

The therapeutic compound (i.e., an antibody or functional part, derivative and/or analogue thereof that comprises a variable domain that binds an extracellular part of EGFR and a variable domain that binds an extracellular part of LGR5) may also be used in conjunction with other well-known therapies (e.g., chemotherapy or radiation therapy) that are selected for their particular usefulness against the cancer that is being treated.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

Preferably, the human subject fulfills any or all of the following requirements
1. Signed informed consent before initiation of any study procedures.
2. Age ≥18 years at signature of informed consent.
3. Histologically or cytologically confirmed solid tumors with evidence of metastatic or locally advanced disease not amenable to standard therapy with curative intent:
Expansion cohort non-CRC tumor types: patients with advanced or metastatic head and neck squamous cell carcinoma may be explored, with or without having been previously treated with at least 2 lines of standard approved therapy.
4. A baseline fresh tumor sample (FFPE and if sufficient material also frozen) from a metastatic or primary site.
5. Amenable for biopsy.
6. Measurable disease as defined by RECIST version 1.1 by radiologic methods.
7. Eastern Cooperative Oncology Group (ECOG) performance status of 0 or 1.
8. Life expectancy ≥12 weeks, as per investigator.
9. Left ventricular ejection fraction (LVEF) ≥50% by echocardiogram (ECHO) or multiple gated acquisition scan (MUGA).
10. Adequate organ function:
   Absolute neutrophil count (ANC) ≥1.5×109/L
   Hemoglobin ≥9 g/dL
   Platelets ≥100×109/L
   Corrected total serum calcium within normal ranges
   Serum magnesium within normal ranges (or corrected with supplements)
   Alanine aminotransferase (ALT), aspartate aminotransferase (AST) ≤2.5×upper limit of normal (ULN) and total bilirubin ≤1.5×ULN (unless due to known Gilbert's syndrome who are excluded if total bilirubin >3.0×ULN or direct bilirubin >1.5×ULN); in cases of liver involvement, ALT/AST ≤5×ULN and total bilirubin ≤2×ULN will be allowed, unless due to known Gilbert's syndrome when total bilirubin ≤3.0×ULN or direct bilirubin ≤1.5×ULN will be allowed or hepatocellular carcinoma [Child-Pugh class A] when total bilirubin <3 mg/dL will be allowed
   Serum creatinine ≤1.5×ULN or creatinine clearance >60 mL/min calculated according to the Cockroft and Gault formula or MDRD formula for patients aged >65 years
   Serum albumin >3.3 g/dL

The compounds and compositions described herein are useful as therapy and in therapeutic treatments and may thus be useful as medicaments and used in a method of preparing a medicament.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate embodiments, however, it will be appreciated that the scope of the invention may include aspects or embodiments having combinations of all or some of the features described.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### EXAMPLES

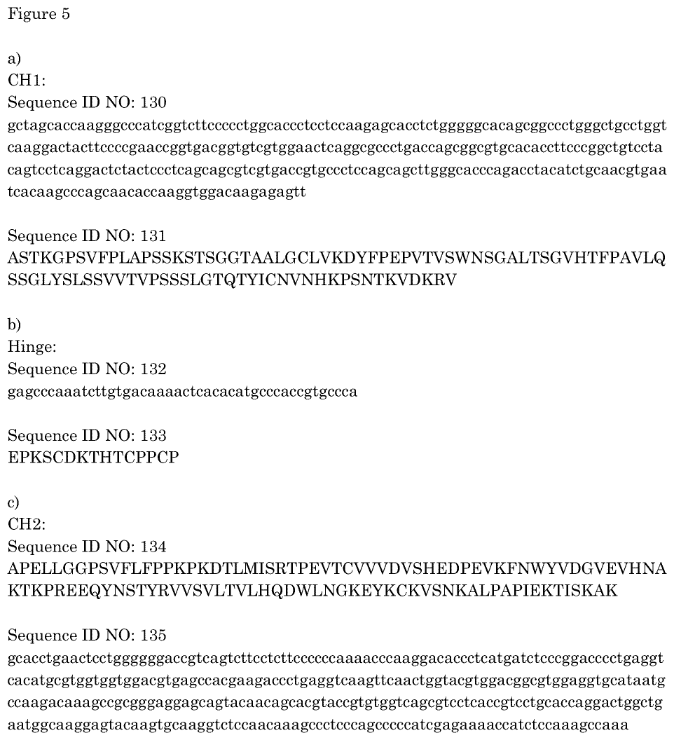

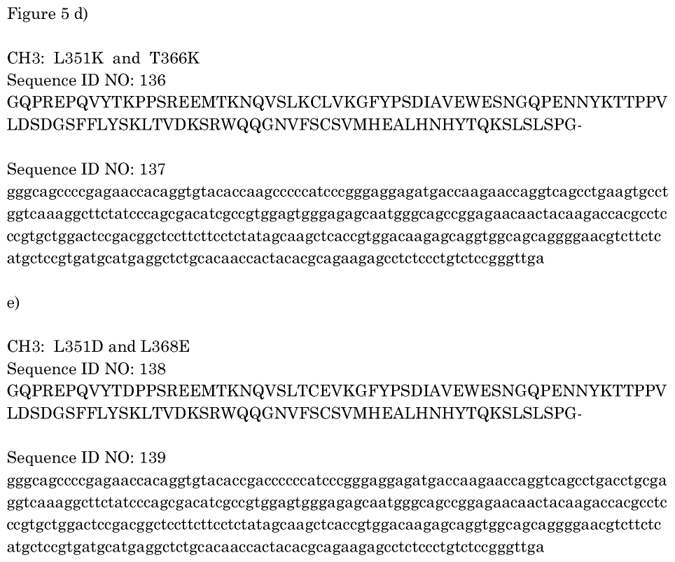

As used herein “MFXXXX” wherein X is independently a numeral 0-9, refers to a Fab comprising a variable domain wherein the VH has the amino acid sequence identified by the 4 digits depicted in FIG. 3. Unless otherwise indicated the light chain variable region of the variable domain typically has a sequence of FIG. 4b. The light chain in the examples has a sequence as depicted in FIG. 4a. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIGS. 5d and 5e. Bispecific antibodies in the examples have an Fc tail with a KK/DE CH3 heterodimerization domain, a CH2 domain and a CH1 domain as indicated in FIG. 5, a common light chain as indicated in FIG. 4a and a VH as specified by the MF number. For example a bispecific antibody indicated by MF3755 xMF5816 has the above general sequences and a variable domain with a VH with the sequence of MF3755 and a variable domain with a VH with the sequence of MF5816.

The amino acid and nucleic acid sequences of the various heavy chain variable regions (VH) are indicated in FIG. 3. Bispecific antibodies EGFR/LGR5, MF3755xMF5816; comprising heavy chain variable regions MF3755 and MF5816 and a common light chain and including modifications for enhanced ADCC from afucosylation, among other LGR5 and EGFR combinations as depicted in FIG. 3 have been shown to be effective in WO2017/069628.

### Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see FIGS. 5d and 5e). It was previously demonstrated in the mentioned applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes of variable domain that bind LGR5 described above were cloned into the vector encoding the positively charged CH3 domain. The VH genes of variable domain that bind EGFR such as those disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0×10e6 cells/ml. Cells were seeded at a density of 0.3-0.5×10e6 viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### IgG Purification and Quantification

Purifications were performed under sterile conditions in filter plates using Protein-A affinity chromatography. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration.

Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

To determine the amount of IgG purified, the concentration of antibody was determined by means of Octet analysis using protein-A biosensors (Forte-Bio, according to the supplier's recommendations) using total human IgG (Sigma Aldrich, cat. nr. I4506) as standard.

The following bispecific antibodies are suitable for use in this example and for use in the methods of the invention: MF3370xMF5790, MF3370x5803, MF3370x5805, MF3370x5808, MF3370x5809, MF3370x5814, MF3370x5816, MF3370x5817, MF3370x5818, MF3755xMF5790, MF3755x5803, MF3755x5805, MF3755x5808, MF3755x5809, MF3755x5814, MF3755x5816, MF3755x5817, MF3755x5818, MF4280xMF5790, MF4280x5803, MF4280x5805, MF4280x5808, MF4280x5809, MF4280x5814, MF4280x5816, MF4280x5817, MF4280x5818, MF4289xMF5790, MF4289x5803, MF4289x5805, MF4289x5808, MF4289x5809, MF4289x5814, MF4289x5816, MF4289x5817, and MF4289x5818. Each bispecific antibody comprises two VH as specified by the MF numbers capable of binding EGFR and LGR5 respectively, further comprises an Fc tail with a KK/DE CH3 heterodimerization domain as indicated by SEQ ID NO: 136 (FIG. 5d) and SEQ ID NO: 138 (FIG. 5e), respectively, a CH2 domain as indicated by SEQ ID NO: 134 (FIG. 5c) and a CH1 domain as indicated by SEQ ID NO: 131 (FIG. 5a), a common light chain as indicated by SEQ ID NO: 121 (FIG. 4).

### Examples 1: In Vivo Evaluation of an Anti-EGFR x Anti-LGR5 Antibody Against Head & Neck Cancer Using Patient-Derived Xenograft (PDX) Mouse Models

### Mice PDX Models

Crown Biosciences Inc. has developed a collection of patient-derived xenograft (PDX) models derived from surgically resected human primary tumors. PDX models as used herein are clinically and molecularly annotated and faithfully represent the clinical epidemiology of the respective tumors. These models can be injected subcutaneously in the flanks of immunodeficient mice. Different head and neck PDX models were used to evaluate the therapeutic efficacy of a full-length IgG1 bispecific antibody comprising MF3755 x MF5816 and further relevant domains as indicated (i.e. CH1, CH2, KK/DE modified CH3 heterodimerization domain and the common light chain). Detailed information of these models, including cancer subtype, presence of genomic mutations, and EGFR/LGR5 expression levels are described in Table 1.

TABLE 1
Characteristics of PDX models originating from Head and Neck cancer patients.

| | Subtype, | | | | | | |
|---|---|---|---|---|---|---|---|
| HN | tissue/ | Mutations | | EGFR Expression | | LGR5 expression | |
| model | organ | * = stop | | | LOG2 | | LOG2 |
| no | involved | X = frameshift | Notes | Fold | (FPKM) | Fold | (FPKM) |
| 2167 | HNSCC, | CDKN2A (RAGAR99- | — | 0.081882332 | 6.32 | 0.001298314 | 2.5422 |
| | larynx | 103X), | | | | | |
| | | CREBBP (R1446C), | | | | | |
| | | CUL1 (D483N), | | | | | |
| | | EPHA3 (S899L), | | | | | |
| | | EXT1 (R723S), | | | | | |
| | | FAT2 (G2421C), | | | | | |
| | | FOXP1 (E581K), | | | | | |
| | | HIST1H3B (R50P), | | | | | |
| | | HSP90AB1 (R456P), | | | | | |
| | | IKZF3 (A311T), | | | | | |
| | | IL6ST (Y554F), | | | | | |
| | | INHBA (R309L), | | | | | |
| | | LMO1 (D47Y), | | | | | |
| | | LPP (M504K), | | | | | |
| | | MSR1 (E107*), | | | | | |
| | | NBN (E564K), | | | | | |
| | | RAD54B (N593S), | | | | | |
| | | RGS3 (R1040Q), | | | | | |
| | | TAOK1 (R504C), | | | | | |
| | | TP53 (R273C), | | | | | |
| | | WNK1 (Y2276*) | | | | | |
| 2590 | HNSCC, | AHR (Q173E), | Ulceration | 0.09062486 | 6.1562 | 1.70936E−05 | −2 |
| | tongue | AHR (E211Q), | | | | | |
| | | ALK (T917I), | | | | | |
| | | ATP6AP2 (S163C), | | | | | |
| | | CDKN2A (D84Y), | | | | | |
| | | EP300 (S1730C), | | | | | |
| | | FGFR1 (R506Q), | | | | | |
| | | FLT4(P1008L), | | | | | |
| | | FN1 (R1496W), | | | | | |
| | | HLA-B (118-119: | | | | | |
| | | TL/TX), | | | | | |
| | | IREB2 (G747E), | | | | | |
| | | MCM8 (G655V), | | | | | |
| | | PLCG2 (R956H), | | | | | |
| | | RB1(E884K), | | | | | |
| | | THRAP3 (S278C), | | | | | |
| | | TP53 (G245S), | | | | | |
| | | WNK1 (724-725: —/X), | | | | | |
| | | YBX1 (E6K), | | | | | |
| | | ZNF638 (splice acceptor | | | | | |
| | | variant) | | | | | |
| 2579 | HNSCC, | DCC (P110L), | Slight BW | 0.072662887 | 6.3068 | 3.88965E−05 | −2 |
| | buccal | DLC1 (R595C), | loss | | | | |
| | mucosa | HRAS (G12S), | | | | | |
| | | LZTS1 (E505K), | | | | | |
| | | SMARCA4 (R1135Q), | | | | | |
| | | WRN (K32R) | | | | | |
| 5124 | HNSCC | APC (R2505Q), | — | 0.086109606 | 5.5114 | 6.1608E−05 | −2 |
| | | ERCC6 (G952D), | | | | | |
| | | MAD1L1 (K349M), | | | | | |
| | | ROS1 (S653F), | | | | | |
| 3164 | ADC, | DLC1 (D1180N), | — | 0.037884885 | 3.5721 | 0.162565065 | 4.4409 |
| | parotid | EPHA4 (P505S), | | | | | |
| | gland | KIAA1549 (E1426K), | | | | | |
| | | MAP2K1 (L375R), | | | | | |
| | | MSH3 (R411H), | | | | | |
| | | TP53 (Y234C) | | | | | |
| | | ECT2L (Q743*), | | | | | |
| 5125 | HNSCC, | ATM (S1383L), | — | 0.033023866 | 5.6525 | 5.32958E−05 | −2 |
| | neck | ECT2L (Q743*), | | | | | |
| | | HLA-B (118-119: | | | | | |
| | | TL/TX), | | | | | |
| | | HLA-B (E69DX), | | | | | |
| | | ITGA9 (R108W), | | | | | |
| | | RB1 (R445*), | | | | | |
| | | RGS3 (R1157L), | | | | | |
| | | SOX17 (L156P), | | | | | |
| | | TP53 (R337C) | | | | | |
| 3411 | HNSCC, | EPHA2 (T511M), | | 0.066758808 | 5.3232 | 0.025256736 | 2.4983 |
| | Larynx | MACF1 (N2198Y), | | | | | |
| | | CUL3 (T467M), | | | | | |
| | | TFEB (V130M), | | | | | |
| | | NOTCH1 (R2087W), | | | | | |
| | | EFTUD2 (R785Q), | | | | | |
| | | SOX9 (D85N), | | | | | |
| | | ARFGAP3 (S317L), | | | | | |
| | | SETD2 (LSK655-657X), | | | | | |
| | | ATR (R109W), | | | | | |
| | | HLA-B (E69DX), | | | | | |
| | | PGR (G580S), | | | | | |
| | | WNK1 (724-725X), | | | | | |
| | | TP53 (QHLIR192-196R), | | | | | |
| 3642 | HNSCC, | EPHA2 (G391R), | | 0.086296399 | 5.3407 | 0.012482772 | 2.3309 |
| | Right neck | AHCTF1 (S880Y), | | | | | |
| | skin | EPHA4 (R514Q), | | | | | |
| | | FAT1 (L3925S), | | | | | |
| | | (K2988I), | | | | | |
| | | FAT1 (Q2775*), | | | | | |
| | | FAT1 (I1069M), | | | | | |
| | | FAT2 (I2501T), | | | | | |
| | | CDKN2A (R58*), | | | | | |
| | | FANCC (E213K), | | | | | |
| | | HSP90AA1 (V597F), | | | | | |
| | | CHD9 (R1481W), | | | | | |
| | | CBFB (R35W), | | | | | |
| | | TP53 (299-300X), | | | | | |
| | | TP53 (I251N), | | | | | |
| | | NF1 (G2502*), | | | | | |
| | | EFTUD2 (N596S), | | | | | |
| | | BCL3 (V284M), | | | | | |
| | | ASXL1 (Q925*), | | | | | |
| | | ARFGAP1 (V184M), | | | | | |
| | | PLXNB2 (S366L), | | | | | |
| | | TAF1 (S1020F), | | | | | |
| | | CSF3R (S130F), | | | | | |
| | | LRP1B (G1637R), | | | | | |
| | | EPHA3 (S532F), | | | | | |
| | | MAP3K13 (D887V), | | | | | |
| | | HLA-B (TL118-119TX), | | | | | |
| | | HLA-B (E69DX), | | | | | |
| | | HDAC9 (E789K), | | | | | |
| | | LEP (P64L), | | | | | |
| | | DLC1 (S1116L), | | | | | |
| | | NOTCH1 (G367S), | | | | | |
| | | KIF5B (P163L), | | | | | |
| | | KIF5B (P163S), | | | | | |
| | | STARD13 (L790F), | | | | | |
| | | ARID4A (H411D), | | | | | |
| | | MYH10 (R169C), | | | | | |
| | | BRWD1 (D408N), | | | | | |
| | | CLTCL1 (R221C), | | | | | |
| | | PLXNB2 (R1578C), | | | | | |
| | | SMARCA1 (R275C) | | | | | |

LGR5 and EGFR expression was determined by RNA sequencing (RNAseq). Mutation status was determined by genomic analysis. HNSCC = Head and neck squamous cell carcinoma; ADC: adenocarcinoma; BW = body weight, FPKM = normalization based on fragments per kilobase of exon model per million mapped reads.

### Tumor Inoculation and Randomization

Fresh tumor tissue for the inoculation was harvested from mice bearing established primary human tumors. The fresh tumors were cut into small pieces (approximately 2-3 mm in diameter) and transplanted subcutaneously at the upper right dorsal flank of the mice. Tumor pieces were inoculated in 6-8 week old female BALB/c Nude or NOD/SCID mice with mean body weight of about 16 to 20 grams. When the mean tumor size reached 100-150 mm3, mice were randomized. A total of 16 mice per model was enrolled in the study (4 control mice and 12 antibody treated mice). The randomization is performed based on “Matched distribution” method (StudyDirector™ software, version 3.1.399.19). Control mice received PBS.

### Treatment and Sampling Schedule

First treatment was given on the day of randomization, which day is considered as day 0 for the experiment. All mice were injected intraperitoneally (i.p.) once a week for 6 weeks using 200 μl injection volume with a dose prepared fresh before dosing from a 20 mg/mL stock solution antibody. Control mice received PBS and antibody-treated mice were treated with adjusted dosing volume for body weight (dosing volume=10 μL/g). Each mouse received 0.5 mg antibody (approx. 25 mg\kg) regardless of their weight, as detailed in Table 2. After the end of the treatment period, all mice had to undergo a 3 week observation period. The observation period was extended if tumors had not grown to the ethically maximum acceptable tumor size. Control mice were injected with PBS using the same injection volume.

TABLE 2
Treatment Plan

| | | | | | [symbol] | | [symbol] |
|---|---|---|---|---|---|---|---|
| | [symbol] | | [symbol] | | [symbol] | [symbol] | [symbol] |
| [symbol] | [symbol] | [symbol] | [symbol] | [symbol] | [symbol] | [symbol] | [symbol] |
| [symbol] | 4 | [symbol] | — | [symbol] | — | [symbol] | [symbol] |
| [symbol] | 4 | [symbol] | [symbol] | [symbol] | [symbol] | [symbol] | [symbol] |
| | | | Dose level | | Dosing Solution | Dose Volume | Dosing |
| Group | Animal No. | Treatment | (mg/kg) | ROA | (mg/mL) | (mL/kg) | Frequency & Duration |
| 1 | 4 | Vehicle | — | i.p. | — | 10 | QW × 6 weeks |
| 2 | 4 | antibody | 25 | i.p. | 2.5 | 10 | QW × 6 weeks |

QW = once a week, ROA = route of administration, i.p. = intraperitoneal.

### Observation, Sample and Data Collection

After tumor inoculation, the animals were checked daily for morbidity and mortality. During routine monitoring the animals were checked for any effects of tumor growth, behavior changes, changes in mobility, food and water consumption, body weight gain/loss (body weights are measured twice per week after randomization), eye/hair matting and any other abnormalities. Mortality and observed clinical signs were recorded for individual animals.

Tumor volumes were measured twice per week post randomization in two dimensions using a caliper, and the volume expressed in mm³ using the formula: V=(L×W×W)/2, where V is tumor volume, L is tumor length (the longest tumor dimension) and W is tumor width (the longest tumor dimension perpendicular to L). The body weights and tumor volumes were recorded by using Study Director™ software (version 3.1.399.19). Mice were sacrificed at the end of week 9 or when the animals reached a humane end point (for example: tumor volume is larger than 2000 mm³ or weight loss is more than 15% of the starting body weight) whichever occurred first.

### Results

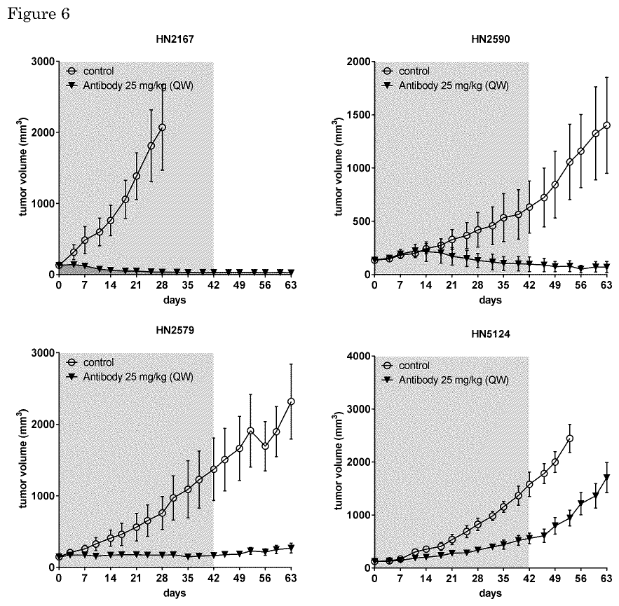

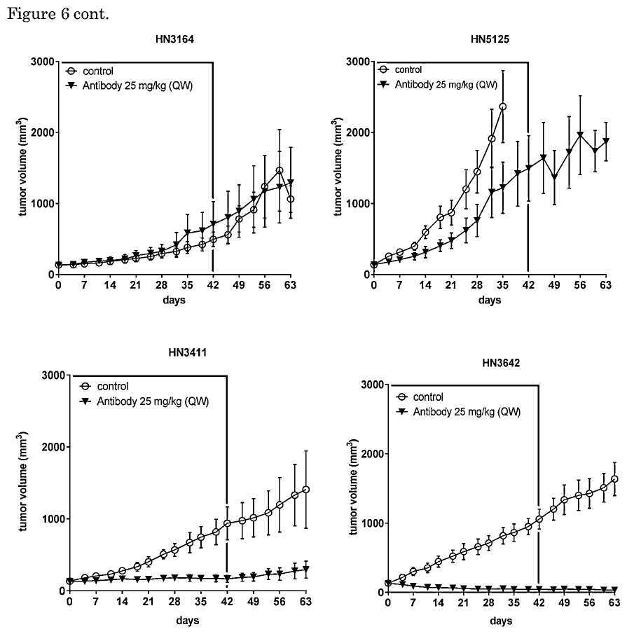

Of the head and neck cancer PDX models tested, treatment with the bispecific antibody showed therapeutic effectivity in seven of the seven head and neck squamous cell carcinomas tested for the period involved (FIG. 6). Also, significantly reduced tumor growth was observed in three models. Models HN2167 and HN2590 showed lower tumor volume at the end of the observation period than at the beginning of the treatment suggesting tumor inhibition mediated by the bispecific antibody in head and neck cancer. Mice of models HN2579, HN5124, HN3642, HN3411 and HN5125 also responded well to antibody treatment and showed a reduction in tumor volume in comparison to vehicle-treated mice.

### Statistical Analysis

To compare tumor volumes of different groups at a pre-specified day, a Bartlett's test was ran first to confirm assumption of homogeneity of variance across all groups. In case the p-value of Bartlett's test is ≥0.05, a one-way ANOVA was run to test overall equality of means across all groups. If the p-value of the one-way ANOVA is <0.05, a further post hoc testing was performed by running Tukey's HSD (honest significant difference) tests for all pairwise comparisons, and Dunnett's tests for comparing each treatment group with the vehicle group. In case the p-value of Bartlett's test is <0.05, a Kruskal-Wallis test was run to test overall equality of medians among all groups. If the p-value the Kruskal-Wallis test is <0.05, a further post hoc testing was performed by running Conover's non-parametric test for all pairwise comparisons or for comparing each treatment group with the vehicle group, both with single-step p-value adjustment. All statistical analyses are done in R—a language and environment for statistical computing and graphics (version 3.3.1). All tests are two-sided unless otherwise specified, and p-values of <0.05 are regarded as statistically significant.

### Example 2: Dose Expansion, and Efficacy of Anti-EGFR x Anti-LGR5 Antibody for Patients Having Head and Neck Cancer

### Phase 1 Dose Escalation Study in Advanced Solid Tumors

### Study Design

A phase 1 open-label multicenter study was performed with an initial dose escalation part to determine the recommended phase 2 dose (RP2D) of an anti-EGFR x anti-LGR5 bispecific antibody of the present disclosure for solid tumors in mCRC patients with a starting dose of 5 mg flat dose. Once the RP2D is established, the antibody is further evaluated in an expansion part of the study, including in patients diagnosed with neck and head cancer. Safety, PK, immunogenicity and preliminary antitumor activity of the antibody is characterized in all patients, and biomarker analyses, including EGFR and LGR5 status is performed.

### Inclusion Criteria

Patients must fulfill all of the following requirements to enter the study:
1. Signed informed consent before initiation of any study procedures.
2. Age ≥18 years at signature of informed consent.
3. Histologically or cytologically confirmed solid tumors with evidence of metastatic or locally advanced disease not amenable to standard therapy with curative intent: Expansion cohort non-CRC tumor types: patients with advanced or metastatic head and neck squamous cell carcinoma may be explored, with or without having been previously treated with at least 2 lines of standard approved therapy.
4. A baseline fresh tumor sample (FFPE and if sufficient material also frozen) from a metastatic or primary site.
5. Amenable for biopsy.
6. Measurable disease as defined by RECIST version 1.1 by radiologic methods.
7. Eastern Cooperative Oncology Group (ECOG) performance status of 0 or 1.
8. Life expectancy ≥12 weeks, as per investigator.
9. Left ventricular ejection fraction (LVEF) ≥50% by echocardiogram (ECHO) or multiple gated acquisition scan (MUGA).
10. Adequate organ function:
   Absolute neutrophil count (ANC) ≥1.5×109/L.
   Hemoglobin ≥9 g/dL
   Platelets ≥100×109/L
   Corrected total serum calcium within normal ranges
   Serum magnesium within normal ranges (or corrected with supplements)
   Alanine aminotransferase (ALT), aspartate aminotransferase (AST) ≤2.5×upper limit of normal (ULN) and total bilirubin ≤1.5×ULN (unless due to known Gilbert's syndrome who are excluded if total bilirubin >3.0×ULN or direct bilirubin >1.5×ULN); in cases of liver involvement, ALT/AST ≤5×ULN and total bilirubin ≤2×ULN will be allowed, unless due to known Gilbert's syndrome when total bilirubin ≤3.0×ULN or direct bilirubin ≤1.5×ULN will be allowed or hepatocellular carcinoma [Child-Pugh class A] when total bilirubin <3 mg/dL will be allowed
   Serum creatinine ≤1.5×ULN or creatinine clearance ≥60 mL/min calculated according to the Cockroft and Gault formula or MDRD formula for patients aged >65 years
   Serum albumin >3.3 g/dL

### Exclusion Criteria

The presence of any of the following criteria excludes a patient from participating in the study:
1. Central nervous system metastases that are untreated or symptomatic, or require radiation, surgery, or continued steroid therapy to control symptoms within 14 days of study entry.
2. Known leptomeningeal involvement.
3. Participation in another clinical trial or treatment with any investigational drug within 4 weeks prior to study entry.
4. Any systemic anticancer therapy within 4 weeks or 5 half-lives whichever is longer of the first dose of study treatment. For cytotoxic agents that have major delayed toxicity (e.g. mitomycin C, nitrosoureas), or anticancer immunotherapies, a washout period of 6 weeks is required.
5. Requirement for immunosuppressive medication (e.g. methotrexate, cyclophosphamide)
6. Major surgery or radiotherapy within 3 weeks of the first dose of study treatment. Patients who received prior radiotherapy to ≥25% of bone marrow are not eligible, irrespective of when it was received.
7. Persistent grade >1 clinically significant toxicities related to prior antineoplastic therapies (except for alopecia); stable sensory neuropathy ≤grade 2 NCI-CTCAE v4.03 is allowed.
8. History of hypersensitivity reaction or any toxicity attributed to human proteins or any of the excipients that warranted permanent cessation of these agents.
9. Uncontrolled hypertension (systolic>150 mmHg and/or diastolic>100 mmHg) with appropriate treatment or unstable angina.
10. History of congestive heart failure of Class II-IV New York Heart Association (NYHA) criteria, or serious cardiac arrhythmia requiring treatment (except atrial fibrillation, paroxysmal supraventricular tachycardia).
11. History of myocardial infarction within 6 months of study entry.
12. History of prior malignancies with the exception of excised cervical intraepithelial neoplasia or non-melanoma skin cancer, or curatively treated cancer deemed at low risk for recurrence with no evidence of disease for at least 3 years.
13. Current dyspnea at rest of any origin, or other diseases requiring continuous oxygen therapy.
14. Patients with a history of interstitial lung disease (e.g.: pneumonitis or pulmonary fibrosis) or evidence of ILD on baseline chest CT scan.
15. Current serious illness or medical conditions including, but not limited to uncontrolled active infection, clinically significant pulmonary, metabolic or psychiatric disorders.
16. Active HIV, HBV, or HCV infection requiring treatment.
17. Patients with current cirrhotic status of Child-Pugh class B or C; known fibrolamellar HCC, sarcomatoid HCC, or mixed cholangiocarcinoma and HCC
18. Pregnant or lactating women; patients of childbearing potential must use highly effective contraception methods prior to study entry, for the duration of study participation, and for 6 months after the last dose of the antibody.

### Dose Escalation

In the dose escalation part, patients with metastatic colorectal cancer (mCRC) adenocarcinoma previously treated in the metastatic setting with standard approved therapy including oxaliplatin, irinotecan and a fluoropyrimidine (5-FU and/or capecitabine), with or without an anti-angiogenic, and an anti-EGFR for KRAS and NRAS wild-type RASwt are treated.

A PK model was generated based on the available bispecific antibody serum concentration data from the preliminary and GLP cynomolgus monkey toxicology studies. Following allometric scaling, this model was used to predict antibody exposure in humans. The antibody starting dose is 5 mg (flat dose) IV, every 2 weeks, with 4-week cycles. Up to 11 dose levels will be investigated: 5, 20, 50, 90, 150, 225, 335, 500, 750, 1100 and 1500 mg (flat dose). The administered dose, dose increments, and frequency of dosing for each patient and each cohort is subject to change based on patient safety, PK and PD data, however the dose will not exceed 4500 mg per cycle.

### Dose-Limiting Toxicity (DLT)

Any of the following clinical toxicities and/or laboratory abnormalities occurring during the first cycle (28 days) and considered by the investigator to be related to antibody treatment will be considered DLT:

### Hematologic Toxicities:

Grade 4 neutropenia (absolute neutrophil count [ANC]<0.5×109 cells/L) for ≥7 days
Grade 3-4 febrile neutropenia
Grade 4 thrombocytopenia
Grade 3 thrombocytopenia associated with bleeding episodes
Other grade 4 hematologic toxicity.
Grade 3-4 non-hematologic AEs and laboratory toxicities with the exception of:
Grade 3-4 infusion-related reactions
Grade 3 skin toxicity that recovers to grade ≤2 within 2 weeks with optimal treatment
Grade 3 diarrhea, nausea and/or vomiting that recover to grade ≤1 or baseline within 3 days with optimal treatment
Grade 3 electrolyte abnormalities that resolve with optimal treatment within 48 hours
Grade 3-4 liver abnormalities lasting ≤48 hours
Any liver function abnormalities that meet the definition of Hy's law.
Any drug-related toxicity lasting >15 days that prevents the next two administrations.

### Dose Expansion

In the expansion part, a bispecific antibody of the present disclosure will be administered at the RP2D in patients having head and neck cancer. Once the RP2D has been defined, additional patients will be treated with this dose and schedule to further characterize safety, tolerability, PK and immunogenicity of antibody, and to perform a preliminary assessment of antitumor activity and biomarker evaluations. The malignancies treated will be known to co-express both targets (i.e. LGR5 and EGFR) and may have prior indication of sensitivity to EGFR inhibition.

Antibody treatment in patients with head and neck cancer will be explored for example, 10 to 20 patients for each indication with potential expansion up to 40 patients, conditional on signs of preliminary anti-tumor activity). The safety of the RP2D will be continuously evaluated during the expansion part of the study by the Safety Monitoring Committee. If the incidence of DLTs exceeds the predefined threshold of 33% for any cohort, enrolment will be paused for this cohort and a full review of the safety, PK, and biomarkers will be performed by the SMC in order to determine if it is safe to continue accrual in that cohort. The overall safety of the drug will also be interrogated at that time.

### Investigational Therapy and Regimen

The anti-EGFR x anti-LGR5 bispecific antibody is formulated as a clear liquid solution for IV infusion. IV infusion is performed every 2 weeks using standard infusion procedures, with a starting dose of 5 mg (flat dose), and with a recommended phase 2 dose of 1500 mg (flat dose). Dose escalation was halted once the RP2D had been reached. Infusions must be administered over a minimum of 4 hours during Cycle 1. Subsequent infusions after Cycle 1 can be reduced to 2 hours at the investigator's discretion and in the absence of IRRs.

### Premedication

During Cycle 1, all infusions will be administered over a period of at least 4 hours with the following premedication regimen: 24 hours before the start of the infusion, 8 mg of dexamethasone PO will be administered 1 hour before the start of the infusion, each patient will receive dexamethasone 20 mg IV, Dexchlorpheniramine 5 mg IV or diphenhydramine 50 mg PO or chlorpheniramine 10 mg IV, Ranitidine 50 mg IV or 150 mg PO and Paracetamol 1 g IV or 650 mg PO.

If a patient tolerates all Cycle 1 infusions with no IRRs and the investigator considers it appropriate, the patient can continue receiving further antibody infusions without accompanying premedication of dexamethasone and the duration of infusions can be reduced to 2 hours. In such cases, the infusion duration may be extended back up to ˜4 hours where considered appropriate to avoid or reduce the incidence or severity of IRRs. For the initial antibody infusion, (Day 1 Cycle 1), each patient will be observed for 6 hours from the start of the infusion, and for 4 hours from the start of the second infusion. Thereafter patients will be observed for the duration of all subsequent administrations (a minimum of 2 hours).

A cycle is considered 4 weeks. For each patient, a 6-hour observation period was implemented following infusion start for the initial antibody infusion, a 4-hour period for the second infusion, and a minimum of 2 hours for all subsequent administrations, corresponding to at least the duration of the infusion. Antibody was administered as a 2 to 4-hour IV infusion every 2 weeks, with 4-week cycles. Day 1 of the subsequent cycle was on Day 29 or after recovery from any adverse effects associated with the previous cycle.

### Treatment Duration

Study treatment is administered until confirmed progressive disease (as per RECIST 1.1), unacceptable toxicity, withdrawal of consent, patient non-compliance, investigator decision (e.g. clinical deterioration), or antibody interruption >6 consecutive weeks. Patients are followed up for safety for at least 30 days following the last antibody infusion and until recovery or stabilization of 5 all related toxicities, and for disease progression and survival status for 12 months.

### Efficacy Assessments

Tumor assessment is based on CT/MRI with contrast per RECIST 1.1 (Eisenhauer et al., 2009 Eur J Cancer 45:228-247), every 8 weeks after treatment start. Objective responses must be confirmed 10 at least 4 weeks after first observation. Bone scans are performed as clinically indicated for patients with bone metastases at baseline or suspected lesions on study. Circulating blood tumor markers, including carcinoembryonic antigen (CEA), are evaluated at screening and on Day 1 of each cycle.

1-19. (canceled)

20. A method of treating head and neck cancer in a subject in need thereof, comprising administering to the subject an antibody or antigen-binding fragment thereof comprising a variable domain that binds an extracellular part of EGFR and a variable domain that binds an extracellular part of LGR5,
wherein the subject is administered a flat dose of 1500 mg of the antibody or an equivalent amount of the antigen-binding fragment thereof,
wherein the variable domain that binds an extracellular part of EGFR comprises a heavy chain variable region comprising the CDR1, CDR2 and CDR3 sequences of a variable region selected from the group consisting of MF3370 (SEQ ID NO: 3); MF3755 (SEQ ID NO: 4); MF4280 (SEQ ID NO: 5) or MF4289 (SEQ ID NO: 6),
wherein the variable domain that binds an extracellular part of LGR5 comprises a heavy chain variable region comprising the CDR1, CDR2 and CDR3 sequences of a variable region selected from the group consisting of MF5790 (SEQ ID NO: 7); MF5803 (SEQ ID NO: 8); MF5805 (SEQ ID NO: 9); MF5808 (SEQ ID NO: 10); MF5809 (SEQ ID NO: 11); MF5814 (SEQ ID NO: 12; MF5816 (SEQ ID NO: 13); MF5817 (SEQ ID NO: 14); or MF5818 (SEQ ID NO: 15) and,
wherein both variable domains comprise CDR1, CDR2 and CDR3 regions of the light chain variable region of SEQ ID NO: 122.

21. The method of claim 20, wherein the subject is a human subject.

22. The method of claim 20, wherein the antibody or antigen-binding fragment thereof is administered intravenously.

23. The method of claim 20, wherein the head and neck cancer is a squamous cell cancer or adenocarcinoma.

24. The method of claim 20, wherein the head and neck cancer is a squamous cell cancer.

25. The method of claim 20, wherein the head and neck cancer is nasopharyngeal cancer, laryngeal cancer, hypopharyngeal cancer, nasal cavity cancer, paranasal sinus cancer, oral cancer or oropharyngeal cancer.

26. The method of claim 20, wherein the head and neck cancer is oropharyngeal squamous cell cancer.

27. The method of claim 20, wherein the cancer is characterized by expression of LGR5 and/or EGFR.

28. The method of claim 20, wherein the variable domain that binds an extracellular part of EGFR comprises a heavy chain variable region comprising the CDR1, CDR2 and CDR3 sequences of the variable region of MF3755 (SEQ ID NO: 4) and wherein the variable domain that binds an extracellular part of LGR5 comprises a heavy chain variable region comprising the CDR1, CDR2 and CDR3 sequences of the variable region of MF5816 (SEQ ID NO: 13).

29. The method of claim 20, wherein a VH chain of the variable domain that binds EGFR comprises the amino acid sequence of VH chain MF3755 (SEQ ID NO: 4); and wherein a VH chain of the variable domain that binds LGR5 comprises the amino acid sequence of VH chain MF5816 (SEQ ID NO: 13).

30. The method of claim 20, wherein a VH chain of the variable domain that binds EGFR comprises the amino acid sequence of VH chain MF3755 (SEQ ID NO: 4); and wherein a VH chain of the variable domain that binds LGR5 comprises the amino acid sequence of VH chain MF5816 (SEQ ID NO: 13).

31. The method of claim 20, wherein both variable domains comprise the light chain variable region of SEQ ID NO: 122.

32. The method of claim 20, wherein the antibody is ADCC enhanced.

33. The method of claim 20, wherein the antibody is afucosylated.


SEQUENCE LISTING (142 sequences)

SEQ ID NO: 1 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 1210 aa, Homo sapiens [sequence omitted]

SEQ ID NO: 3 - protein, 122 aa, Artificial Sequence
  VH 3370
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 4 - protein, 120 aa, Artificial Sequence
  VH 3755
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 5 - protein, 125 aa, Artificial Sequence
  VH 4280
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 6 - protein, 119 aa, Artificial Sequence
  VH 4289
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 7 - protein, 126 aa, Artificial Sequence
  VH 5790
QVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 8 - protein, 126 aa, Artificial Sequence
  VH 5803
QVQLQESGPGLVKPSETLSLTCTVSNGSISTYYWSWIRQPPGKGLEWIGYVYYTGRTKYNPSLKSRVTISVDTSKNQFSLNLSSVTAADTAVYYCARGGIVVVPAARDYYYYMDVWGKGTTVTVSS

SEQ ID NO: 9 - protein, 120 aa, Artificial Sequence
  VH 5805
EVQLVQSGAEVKKPGESLKIACKGSGFSFTSHWIGWVRQKPGRGLEWMGVIYPGDSDTRYSPSFQGQVTVSADKSINTAYLQWNSLKASDTAIYYCARPNSGSPRYFEFWGRGTLVTVSS

SEQ ID NO: 10 - protein, 126 aa, Artificial Sequence
  VH 5808
QVQLQESGPGLVKPSETLSLTCTVSGGSISSSSYYWGWIRQPPGKGLEWIGSFYYSGNTYYNPSLKSRVTISVDTSKNQFSLKLSSVTAADTAVYYCARQEYYYGSGSPSYYFDYWGQGTLVTVSS

SEQ ID NO: 11 - protein, 120 aa, Artificial Sequence
  VH 5809
EVQLVQSGAEVKKPGESLKISCKGSGDSFISHWIAWVRQMPGKGLEWMGIVYPGDSDTRYSPSFQGQVTISADKSITTAYLQWSSLKASDTAMYYCARHEWELLGPFDYWGQGTLVTVSS

SEQ ID NO: 12 - protein, 121 aa, Artificial Sequence
  VH 5814
EVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 13 - protein, 127 aa, Artificial Sequence
  VH 5816
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 14 - protein, 119 aa, Artificial Sequence
  VH 5817
QVQLVQSGAEVKKPGSSVKVSCKVSGGTFRSYAISWVRQAPGQGLEWMGGIIPIFDTRNYAQILQGRVTITADLSTSTAYMELNSLRSEDTAIYYCARGSDEGDWFDPWGQGTLVTVSS

SEQ ID NO: 15 - protein, 121 aa, Artificial Sequence
  VH 5818
EVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSS

SEQ ID NO: 16 - protein, 30 aa, Artificial Sequence
  FW1 3370
QVQLVQSGAEVKKPGASVKVSCKASGYTFT

SEQ ID NO: 17 - protein, 5 aa, Artificial Sequence
  CDR1 3370
SYGIS

SEQ ID NO: 18 - protein, 14 aa, Artificial Sequence
  FW2 3370
WVRQAPGQGLEWMG

SEQ ID NO: 19 - protein, 17 aa, Artificial Sequence
  CDR2 3370
WISAYNGNTNYAQKLQG

SEQ ID NO: 20 - protein, 32 aa, Artificial Sequence
  FW3 3370
RVTMTTDTSTSTAYMELRSLRSDDTAVYYCAK

SEQ ID NO: 21 - protein, 13 aa, Artificial Sequence
  CDR3 3370
DRHWHWWLDAFDY

SEQ ID NO: 22 - protein, 11 aa, Artificial Sequence
  FW4 3370
WGQGTLVTVSS

SEQ ID NO: 23 - protein, 30 aa, Artificial Sequence
  FW1 3755
QVQLVQSGSELKKPGASVKISCKASGYDFT

SEQ ID NO: 24 - protein, 5 aa, Artificial Sequence
  CDR1 3755
NYAMN

SEQ ID NO: 25 - protein, 14 aa, Artificial Sequence
  FW2 3755
WVRQAPGHGLEWMG

SEQ ID NO: 26 - protein, 17 aa, Artificial Sequence
  CDR2 3755
WINANTGDPTYAQGFTG

SEQ ID NO: 27 - protein, 32 aa, Artificial Sequence
  FW3 3755
RFVFSLDTSVSTAYLQISSLKAEDSAVYYCTR

SEQ ID NO: 28 - protein, 11 aa, Artificial Sequence
  CDR3 3755
ERFLEWLHFDY

SEQ ID NO: 29 - protein, 11 aa, Artificial Sequence
  FW4 3755
WGQGTLVTVSS

SEQ ID NO: 30 - protein, 30 aa, Artificial Sequence
  FW1 4280
QVQLVQSGAEVKKPGASVKVSCKVSGYTLT

SEQ ID NO: 31 - protein, 5 aa, Artificial Sequence
  CDR1 4280
ELSMH

SEQ ID NO: 32 - protein, 14 aa, Artificial Sequence
  FW2 4280
WVRQAPGKGLEWMG

SEQ ID NO: 33 - protein, 17 aa, Artificial Sequence
  CDR2 4280
GFDPEYGKTFFAQNFQG

SEQ ID NO: 34 - protein, 32 aa, Artificial Sequence
  FW3 4280
RVTMTEDTSADTAYMELSSLRSEDTAVYYCAT

SEQ ID NO: 35 - protein, 16 aa, Artificial Sequence
  CDR3 4280
EGYYETTTYYYNLFDS

SEQ ID NO: 36 - protein, 11 aa, Artificial Sequence
  FW4 4280
WGQGTLVTVSS

SEQ ID NO: 37 - protein, 30 aa, Artificial Sequence
  FW1 4289
QVQLVQSGSELKKPGASVKVSCKTSGYTFT

SEQ ID NO: 38 - protein, 5 aa, Artificial Sequence
  CDR1 4289
DYAMT

SEQ ID NO: 39 - protein, 14 aa, Artificial Sequence
  FW2 4289
WVRQAPGQGLEWMG

SEQ ID NO: 40 - protein, 17 aa, Artificial Sequence
  CDR2 4289
WITTNTGDPTYAPGFTG

SEQ ID NO: 41 - protein, 32 aa, Artificial Sequence
  FW3 4289
RFVFSLDTSVSTAYLQISSLKAEDTAVYYCAR

SEQ ID NO: 42 - protein, 10 aa, Artificial Sequence
  CDR3 4289
VYHWIRGFEF

SEQ ID NO: 43 - protein, 11 aa, Artificial Sequence
  FW4 4289
WGQGTLVTVSS

SEQ ID NO: 44 - protein, 30 aa, Artificial Sequence
  FW1 5790
QVQLQESGPGLVKPSETLSLTCTVSGGSFS

SEQ ID NO: 45 - protein, 7 aa, Artificial Sequence
  CDR1 5790
SSSSYWG

SEQ ID NO: 46 - protein, 14 aa, Artificial Sequence
  FW2 5790
WIRQPPGKGLEWIG

SEQ ID NO: 47 - protein, 16 aa, Artificial Sequence
  CDR2 5790
SFYYSGNTYYNPSLKS

SEQ ID NO: 48 - protein, 32 aa, Artificial Sequence
  FW3 5790
RVTISEDTSKNQFSLKLSSVTAADTAVYYCAR

SEQ ID NO: 49 - protein, 16 aa, Artificial Sequence
  CDR3 5790
QTYSSSWDGVLYYFDY

SEQ ID NO: 50 - protein, 11 aa, Artificial Sequence
  FW4 5790
WGQGTLVTVSS

SEQ ID NO: 51 - protein, 30 aa, Artificial Sequence
  FW1 5803
QVQLQESGPGLVKPSETLSLTCTVSNGSIS

SEQ ID NO: 52 - protein, 5 aa, Artificial Sequence
  CDR1 5803
TYYWS

SEQ ID NO: 53 - protein, 14 aa, Artificial Sequence
  FW2 5803
WIRQPPGKGLEWIG

SEQ ID NO: 54 - protein, 16 aa, Artificial Sequence
  CDR2 5803
YVYYTGRTKYNPSLKS

SEQ ID NO: 55 - protein, 32 aa, Artificial Sequence
  FW3 5803
RVTISVDTSKNQFSLNLSSVTAADTAVYYCAR

SEQ ID NO: 56 - protein, 18 aa, Artificial Sequence
  CDR3 5803
GGIVVVPAARDYYYYMDV

SEQ ID NO: 57 - protein, 11 aa, Artificial Sequence
  FW4 5803
WGKGTTVTVSS

SEQ ID NO: 58 - protein, 30 aa, Artificial Sequence
  FW1 5805
EVQLVQSGAEVKKPGESLKIACKGSGFSFT

SEQ ID NO: 59 - protein, 5 aa, Artificial Sequence
  CDR1 5805
SHWIG

SEQ ID NO: 60 - protein, 14 aa, Artificial Sequence
  FW2 5805
WVRQKPGRGLEWMG

SEQ ID NO: 61 - protein, 17 aa, Artificial Sequence
  CDR2 5805
VIYPGDSDTRYSPSFQG

SEQ ID NO: 62 - protein, 32 aa, Artificial Sequence
  FW3 5805
QVTVSADKSINTAYLQWNSLKASDTAIYYCAR

SEQ ID NO: 63 - protein, 11 aa, Artificial Sequence
  CDR3 5805
PNSGSPRYFEF

SEQ ID NO: 64 - protein, 11 aa, Artificial Sequence
  FW4 5805
WGRGTLVTVSS

SEQ ID NO: 65 - protein, 30 aa, Artificial Sequence
  FW1 5808
QVQLQESGPGLVKPSETLSLTCTVSGGSIS

SEQ ID NO: 66 - protein, 7 aa, Artificial Sequence
  CDR1 5808
SSSYYWG

SEQ ID NO: 67 - protein, 14 aa, Artificial Sequence
  FW2 5808
WIRQPPGKGLEWIG

SEQ ID NO: 68 - protein, 16 aa, Artificial Sequence
  CDR2 5808
SFYYSGNTYYNPSLKS

SEQ ID NO: 69 - protein, 32 aa, Artificial Sequence
  FW3 5808
RVTISVDTSKNQFSLKLSSVTAADTAVYYCAR

SEQ ID NO: 70 - protein, 16 aa, Artificial Sequence
  CDR3 5808
QEYYYGSGSPSYYFDY

SEQ ID NO: 71 - protein, 11 aa, Artificial Sequence
  FW4 5808
WGQGTLVTVSS

SEQ ID NO: 72 - protein, 30 aa, Artificial Sequence
  FW1 5809
EVQLVQSGAEVKKPGESLKISCKGSGDSFI

SEQ ID NO: 73 - protein, 5 aa, Artificial Sequence
  CDR1 5809
SHWIA

SEQ ID NO: 74 - protein, 14 aa, Artificial Sequence
  FW2 5809
WVRQMPGKGLEWMG

SEQ ID NO: 75 - protein, 17 aa, Artificial Sequence
  CDR2 5809
IVYPGDSDTRYSPSFQG

SEQ ID NO: 76 - protein, 32 aa, Artificial Sequence
  FW3 5809
QVTISADKSITTAYLQWSSLKASDTAMYYCAR

SEQ ID NO: 77 - protein, 11 aa, Artificial Sequence
  CDR3 5809
HEWELLGPFDY

SEQ ID NO: 78 - protein, 11 aa, Artificial Sequence
  FW4 5809
WGQGTLVTVSS

SEQ ID NO: 79 - protein, 30 aa, Artificial Sequence
  FW1 5814
EVQLVQSGAEVKKPGSSVKVSCKASGGTST

SEQ ID NO: 80 - protein, 5 aa, Artificial Sequence
  CDR1 5814
NDAIS

SEQ ID NO: 81 - protein, 14 aa, Artificial Sequence
  FW2 5814
WVRQTPGQGLEWMG

SEQ ID NO: 82 - protein, 17 aa, Artificial Sequence
  CDR2 5814
SIIPILDTTDHAQKFQG

SEQ ID NO: 83 - protein, 32 aa, Artificial Sequence
  FW3 5814
RVTITADKSTNTAYMELNSLRSDDTAVYYCAR

SEQ ID NO: 84 - protein, 12 aa, Artificial Sequence
  CDR3 5814
EHIAARQDYFDY

SEQ ID NO: 85 - protein, 11 aa, Artificial Sequence
  FW4 5814
WGQGTLVTVSS

SEQ ID NO: 86 - protein, 30 aa, Artificial Sequence
  FW1 5816
EVQLVQSGSKLKKPGASVKVSCKASGYTFT

SEQ ID NO: 87 - protein, 5 aa, Artificial Sequence
  CDR1 5816
SYTMN

SEQ ID NO: 88 - protein, 14 aa, Artificial Sequence
  FW2 5816
WVRQAPGQGLEWMG

SEQ ID NO: 89 - protein, 17 aa, Artificial Sequence
  CDR2 5816
WINTDTGDPTYAQGFTG

SEQ ID NO: 90 - protein, 32 aa, Artificial Sequence
  FW3 5816
RFVFSLDTSVSTAFLQINSLKAEDTAVYYCAR

SEQ ID NO: 91 - protein, 18 aa, Artificial Sequence
  CDR3 5816
GDCDSTSCYRYSYGYEDY

SEQ ID NO: 92 - protein, 11 aa, Artificial Sequence
  FW4 5816
WGQGTLVTVSS

SEQ ID NO: 93 - protein, 30 aa, Artificial Sequence
  FW1 5817
QVQLVQSGAEVKKPGSSVKVSCKVSGGTFR

SEQ ID NO: 94 - protein, 5 aa, Artificial Sequence
  CDR1 5817
SYAIS

SEQ ID NO: 95 - protein, 14 aa, Artificial Sequence
  FW2 5817
WVRQAPGQGLEWMG

SEQ ID NO: 96 - protein, 17 aa, Artificial Sequence
  CDR2 5817
GIIPIFDTRNYAQILQG

SEQ ID NO: 97 - protein, 32 aa, Artificial Sequence
  FW3 5817
RVTITADLSTSTAYMELNSLRSEDTAIYYCAR

SEQ ID NO: 98 - protein, 10 aa, Artificial Sequence
  CDR3 5817
GSDEGDWFDP

SEQ ID NO: 99 - protein, 11 aa, Artificial Sequence
  FW4 5817
WGQGTLVTVSS

SEQ ID NO: 100 - protein, 30 aa, Artificial Sequence
  FW1 5818
EVQLVQSGTEVRKPGSSVKVSCKASGGTFS

SEQ ID NO: 101 - protein, 5 aa, Artificial Sequence
  CDR1 5818
NYAIS

SEQ ID NO: 102 - protein, 14 aa, Artificial Sequence
  FW2 5818
WVRQAPGQGLEWMG

SEQ ID NO: 103 - protein, 17 aa, Artificial Sequence
  CDR2 5818
SIIPILGTTDHAQKFQD

SEQ ID NO: 104 - protein, 32 aa, Artificial Sequence
  FW3 5818
RVTITADKSSNTTYMELSSLRSDDTAVYYCAR

SEQ ID NO: 105 - protein, 12 aa, Artificial Sequence
  CDR3 5818
EYIAARLDYFDS

SEQ ID NO: 106 - protein, 11 aa, Artificial Sequence
  FW4 5818
WGQGTLVTVSS

SEQ ID NO: 107 - DNA, 381 bp, Artificial Sequence
  MF1337
gaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagt

SEQ ID NO: 108 - DNA, 366 bp, Artificial Sequence
  MF3370
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacgcctttgattattggggccaaggtaccctggtcaccgtctccagt

SEQ ID NO: 109 - DNA, 360 bp, Artificial Sequence
  MF3755
caggtgcagctggtgcagtctgggtctgagttgaagaagcctggggcctcagtgaagatttcctgcaaggcttctggatacgacttcactaactatgctatgaattgggtgcgacaggcccctggacacgggcttgagtggatgggatggatcaacgccaacactggggacccaacgtatgcccagggcttcacaggacggtttgtcttctccttggacacctctgtcagcacggcatatctgcagatcagcagtttaaaggctgaggactctgccgtgtattactgtacgagagagcgatttttggagtggttacactttgactactggggccagggaaccctggtcaccgtctccagt

SEQ ID NO: 110 - DNA, 375 bp, Artificial Sequence
  MF4280
caggtgcagctggtgcagtctggggctgaggtgaagaagcctggggcctcagtgaaggtctcctgcaaggtttccggatacaccctcactgaattatccatgcactgggtgcgacaggctcctggtaaagggcttgaatggatgggaggctttgatcctgagtatggtaaaacattcttcgcacagaacttccagggcagagtcaccatgaccgaggacacatctgcagacacagcctacatggagctaagcagcctgagatctgaggacacggccgtgtattactgtgcaacagaggggtattatgagactactacttattactacaacctttttgactcctggggccagggaaccctggtcaccgtctccagt

SEQ ID NO: 111 - DNA, 357 bp, Artificial Sequence
  MF4289
caggtgcagctggtgcaatctgggtctgaattgaagaagcctggggcctcagtgaaggtttcctgcaagacttctggatacaccttcactgactatgctatgacttgggtgcgacaggcccctggacaagggcttgaatggatgggatggatcaccaccaacactggggacccaacgtatgccccgggcttcacaggacggtttgtcttctccttggacacctctgtcagcacggcatatctgcagatcagcagcctaaaggccgaggacactgccgtatattactgtgcgagagtgtatcattggatacggggatttgagttttggggccagggaaccctggtcaccgtctccagt

SEQ ID NO: 112 - DNA, 378 bp, Artificial Sequence
  MF5790
caggtgcagctgcaggagtcgggcccaggactggtgaagccttcggagaccctgtccctcacctgcactgtctctggtggctccttcagcagtagtagttcctactggggctggatccgccagcccccagggaaggggctggagtggattgggagtttctattatagtgggaacacctactacaacccgtccctcaagagtcgagtcaccatatccgaagacacgtccaagaaccagttctccctgaagctgagctctgtgaccgccgcagacacggctgtgtattactgtgcgagacagacgtatagcagcagctgggacggggtcctgtactactttgactactggggccagggaaccctggtcaccgtctccagt

SEQ ID NO: 113 - DNA, 378 bp, Artificial Sequence
  MF5803
caggtgcagctgcaggagtcggggccaggactggtgaagccttcggagaccctgtccctcacctgcactgtctctaatggctccatcagtacttactactggagctggatccggcagcccccagggaaggggctggagtggattggatatgtctattacactgggcgcaccaagtacaacccctccctcaagagtcgagtcaccatatcagtagacacgtccaagaaccagttctccctgaacctgagttctgtgaccgctgcggacacggccgtgtattactgtgcgagagggggtattgtagtagtcccagctgcgcgggactattactactacatggacgtctggggcaaagggaccacggtcaccgtctccagt

SEQ ID NO: 114 - DNA, 360 bp, Artificial Sequence
  MF5805
gaggtgcaactggtgcagtctggagcagaggtgaaaaagcccggggagtctctgaagatcgcctgtaagggttctggattcagttttaccagccactggatcggctgggtgcgccagaagcccgggagaggcctggagtggatgggggtcatctatcctggtgactctgataccagatacagcccgtccttccaaggccaggtcaccgtctcagccgacaagtccatcaataccgcctacctgcagtggaacagcctgaaggcctcggacaccgccatatattactgtgcgagaccgaacagtgggagtccccggtacttcgagttctggggccgtggcaccctggtcaccgtctccagt

SEQ ID NO: 115 - DNA, 378 bp, Artificial Sequence
  MF5808
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtttctattatagtgggaacacctactacaacccgtccctcaagagtcgagtcaccatatccgtagacacgtccaagaaccagttctccctgaagctgagctctgtgaccgccgcagacacggctgtgtattactgtgcgagacaggagtattactatggttcggggagtccttcgtactactttgactactggggccagggaaccctggtcaccgtctccagt

SEQ ID NO: 116 - DNA, 360 bp, Artificial Sequence
  MF5809
gaggtgcagctggtgcagtctggagcagaggtgaaaaagcccggggagtctctgaagatctcctgtaagggttctggagacagttttatcagccactggatcgcctgggtgcgccagatgcccgggaaaggcctggagtggatggggatcgtctatcctggtgactctgataccagatacagcccgtccttccaaggccaggtcaccatctcagccgacaagtccatcaccaccgcctacttgcagtggagcagcctgaaggcctcggacaccgccatgtattactgtgcgagacacgagtgggaactacttggcccctttgactactggggccagggaaccctggtcaccgtctccagt

SEQ ID NO: 117 - DNA, 363 bp, Artificial Sequence
  MF5814
gaggtgcagctggtgcagtctggggctgaggtgaagaagcctgggtcctcggtgaaggtctcctgcaaggcttctggaggcacctccactaacgatgctatcagttgggtgcgacagacccctggacaagggcttgagtggatgggaagtatcatccctatccttgatacaacagaccacgcacagaagttccagggcagagtcacgattaccgcggacaaatccacgaacacagcctacatggagctgaacagcctgagatctgatgacacggccgtgtattactgtgcgagagagcatatagcagctcgtcaggactactttgactattggggccagggaaccctggtcaccgtctccagt

SEQ ID NO: 118 - DNA, 381 bp, Artificial Sequence
  MF5816
gaggtgcagctggtgcagtctgggtctaaattgaagaagcctggggcctcagtgaaggtttcctgcaaggcttctggatacaccttcactagctatactatgaattgggtgcgacaggcccctggacaagggcttgagtggatgggatggatcaacaccgacactggggacccaacgtatgcccagggcttcacaggacggtttgtcttctccttggacacctctgtcagcacggcatttctacagatcaacagcctaaaggctgaggacactgccgtatattactgtgcgagaggagattgtgatagtaccagctgctatagatacagttatggttacgaggactactggggccagggaaccctggtcaccgtctccagt

SEQ ID NO: 119 - DNA, 357 bp, Artificial Sequence
  MF5817
caggtgcagctggtgcagtctggggctgaggtgaagaagcctgggtcctcggtgaaggtctcctgcaaggtttctggaggcaccttcaggagctatgctatcagctgggtgcgacaggcccctggacaagggcttgagtggatgggagggatcatccctatctttgatacaagaaactacgcacagattcttcagggcagagtcacgattaccgcggacttatccacgagcacagcctacatggagctgaacagtctgagatctgaggacacggccatttattactgtgcgagagggagcgacgagggggactggttcgacccctggggccaaggaaccctggtcaccgtctccagt

SEQ ID NO: 120 - DNA, 363 bp, Artificial Sequence
  MF5818
gaggtgcagctggtgcagtctgggactgaggtgaggaagcctgggtcctcggtgaaggtctcctgcaaggcttctggaggcaccttcagcaactatgctatcagctgggtgcgacaggcccctggacaggggcttgagtggatgggaagtatcatccctatccttggaacaacagaccacgcacagaagttccaggacagagtcacgattaccgcggacaaatcctcgaacacaacctacatggagctgagcagcctgagatctgatgacacggccgtatattactgtgcgagagagtatatagcagctcgtctggactactttgactcttggggccagggaaccctggtcaccgtctccagt

SEQ ID NO: 121 - protein, 214 aa, Artificial Sequence
  common light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 107 aa, Artificial Sequence
  IGKV1-39/jk1 variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 123 - DNA, 321 bp, Artificial Sequence
  IGKV1-39/jk1 variable region
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 124 - protein, 107 aa, Artificial Sequence [sequence omitted]

SEQ ID NO: 125 - DNA, 320 bp, Artificial Sequence
  light chain constant region
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 126 - protein, 95 aa, Artificial Sequence
  V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 127 - protein, 6 aa, Artificial Sequence
  CDR1 LC
QSISSY

SEQ ID NO: 128 - protein, 3 aa, Artificial Sequence
  CDR2 LC
AAS

SEQ ID NO: 129 - protein, 9 aa, Artificial Sequence
  CDR3 LC
QQSYSTPPT

SEQ ID NO: 130 - DNA, 294 bp, Artificial Sequence
  CH1
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 131 - protein, 98 aa, Artificial Sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 132 - DNA, 45 bp, Artificial Sequence
  hinge
gagcccaaatcttgtgacaaaactcacacatgcccaccgtgccca

SEQ ID NO: 133 - protein, 15 aa, Artificial Sequence
  hinge
EPKSCDKTHTCPPCP

SEQ ID NO: 134 - protein, 110 aa, Artificial Sequence
  CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 135 - DNA, 330 bp, Artificial Sequence
  CH2
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 136 - protein, 106 aa, Artificial Sequence
  CH3 L351K  and  T366K
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - DNA, 321 bp, Artificial Sequence
  CH3 L351K  and  T366K
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 138 - protein, 106 aa, Artificial Sequence
  CH3  L351D  and  L368E
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 139 - DNA, 321 bp, Artificial Sequence
  CH3  L351D  and  L368E
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 140 - protein, 7 aa, Artificial Sequence
  CDR3 LC
QQSYSTP

SEQ ID NO: 141 - DNA, 14 bp, Homo sapiens
gggccggggcgcgg

SEQ ID NO: 142 - protein, 5 aa, Homo sapiens
RAGAR